(19) United States
(12) Patent Application Publication
(10) Pub. No.: US 2023/0190286 A1
(43) Pub. Date: Jun. 22, 2023

(54) IMPLANTABLE SPHINCTER ASSISTANCE DEVICE WITH INTRALUMINAL INTRODUCTION, EXTRALUMINAL DEPLOYMENT, AND EXTRALUMINAL SECURING

(71) Applicant: Cilag GmbH International, Zug (CH)

(72) Inventors: Frederick E. Shelton, IV, Hillsboro, OH (US); Kevin M. Fiebig, Cincinnati, OH (US)

(21) Appl. No.: 17/552,522

(22) Filed: Dec. 16, 2021

(51) Int. Cl. A61B 17/12; A61B 17/34; A61B 1/05; A61B 1/273; A61B 17/04

(52) U.S. Cl. CPC A61B 17/12013; A61B 17/3415; A61B 1/05; A61B 1/2733; A61B 17/0469

(57) ABSTRACT

An apparatus includes a first shaft extending distally from a proximal end to a distal end. The first shaft is sized to fit within an esophagus of a patient. The first shaft includes a first shaft lumen extending distally to the distal end. The apparatus further includes a second shaft slidably positioned within the first shaft lumen. The second shaft is sized to fit within the shaft lumen and extend through the transverse bore. The second shaft is configured to receive a guide element and a sphincter augmentation device so that the sphincter augmentation device may be deployed through an interior of the esophagus to an exterior of the esophagus.

### BACKGROUND

In some instances, it may be desirable to place a medical implant within or surrounding a biological lumen/passageway in order to improve or assist the function of, or otherwise affect, the biological lumen/passageway. Examples of such biological lumens/passageways include, but are not limited to, the esophagus, a fallopian tube, a urethra, or a blood vessel. Some biological passages normally function by expanding and contracting actively or passively to regulate the flow of solids, liquids, gasses, or a combination thereof. The ability of a biological passage to expand and contract may be compromised by defects or disease. One merely illustrative example of a condition associated with decreased functionality of a body passage is Gastro Esophageal Reflux Disease (“GERD”), which effects the esophagus.

A normal, heathy, esophagus is a muscular tube that carries food from the mouth, through the chest cavity and into the upper part of the stomach. A small-valved opening in the esophagus, called the lower esophageal sphincter (“LES”), regulates the passage of food from the esophagus into the stomach, as well as the passage of acidic fluids and food from the stomach toward the esophagus. The LES may also regulate stomach intra-gastric pressures. A healthy LES may contain pressure of gasses within the stomach at around 10 mm Hg greater than normal intragastrical pressure, thereby impeding acidic gases/fluids from refluxing from the stomach back into the esophagus. When functioning properly, a pressure difference greater than 10 mm Hg may regulate when the LES opens to allow gasses to be vented from the stomach toward the esophagus.

If the LES relaxes, atrophies, or degrades for any reason, the LES may cease functioning properly. Therefore, the LES may fail to sufficiently contain pressure of gasses within the stomach such that acidic contents of the stomach may travel back into the esophagus, resulting in reflux symptoms. Two primary components that control the LES are the intrinsic smooth muscle of the distal esophagus wall and the skeletal muscle of the crural diaphragm or esophageal hiatus. A causation of esophageal reflux, which may be associated with GERD, is relaxation of one or both of the smooth muscle of the distal esophagus wall or the hiatal diaphragm sphincter mechanisms. Chronic or excessive acid reflux exposure may cause esophageal damage. Conventionally, treatment for GERD may involve either open or endoscopic surgical procedures. Some procedures may include a fundoplication that mobilizes the stomach relative to the lower esophagus; or suturing a pleat of tissue between the LES and the stomach to make the lower esophagus tighter.

Examples of devices and methods that have been developed to treat anatomical lumens by providing sphincter augmentation are described in U.S. Pat. No. 7,175,589, entitled “Methods and Devices for Luminal and Sphincter Augmentation,” issued Feb. 13, 2007, the disclosure of which is incorporated by reference herein, in its entirety; U.S. Pat. No. 7,695,427, entitled “Methods and Apparatus for Treating Body Tissue Sphincters and the Like,” issued Apr. 13, 2010, the disclosure of which is incorporated by reference herein, in its entirety; U.S. Pat. No. 8,070,670, entitled “Methods and Devices for Luminal and Sphincter Augmentation,” issued Dec. 6, 2011, the disclosure of which is incorporated by reference herein, in its entirety; and U.S. Pat. No. 8,734,475, entitled “Medical Implant with Floating Magnets,” issued May 27, 2014, the disclosure of which is incorporated by reference herein, in its entirety.

While various kinds and types of instruments have been made and used to treat or otherwise engage anatomical lumens, it is believed that no one prior to the inventors has made or used an invention as described herein.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1 depicts a cross-sectional side view, taken along a coronal plane of the body, of a biological passage;

FIG. 2 depicts a cross-sectional isometric view, taken along a coronal plane of the body, of a human esophago-gastric junction;

FIG. 3 depicts a top plan view of an example of a sphincter augmentation device;

FIG. 4 depicts a partial, cross-sectional view of a portion of the sphincter augmentation device of FIG. 3;

FIG. 5A depicts a top, cross-sectional view of the sphincter augmentation device of FIG. 3 positioned about an LES, with the sphincter augmentation device in an open and expanded configuration;

FIG. 5B depicts a top, cross-sectional view of the sphincter augmentation device of FIG. 3 positioned about the LES of FIG. 5A, with the sphincter augmentation device in a closed and contracted configuration;

FIG. 6 depicts a top, a cross-sectional view of another sphincter augmentation device including a plurality of beads, with a plurality of magnetic balls positioned within housings of the beads;

FIG. 7 depicts a perspective view of a single bead of the plurality of beads shown in FIG. 6, with the outer housing shown in phantom for additional clarity;

FIG. 8A depicts a cross-sectional view of a bead of the plurality of beads shown in FIG. 6 within range of a machine configured to produce a magnetic field, with the machine in a non-activated state and the magnetic balls positioned in a default position;

FIG. 8B depicts a cross-sectional view of the bead and machine of FIG. 8A, with the machine in an activated state producing a magnetic field and the magnetic balls oriented in an aligned position relative to the magnetic field;

FIG. 8C depicts a cross-sectional view of the bead and machine of FIG. 8A, with the machine oriented within range at an alternate position relative to the bead, with the machine in the activated state and the magnetic balls positioned in another aligned position relative to the magnetic field;

FIG. 9A depicts a top plan view of the sphincter augmentation device of FIG. 6, with the beads in default position, without an external magnetic field acting on the magnets, with some of the beads shown in cross-section;

FIG. 9B depicts a top plan view the sphincter augmentation device of FIG. 6, with the beads in an aligned position, with an external magnetic field acting on the magnets;

FIG. 10 depicts a cross-sectional view of another pair of beads for use with the sphincter augmentation device of FIG. 6, each bead including a sidewall having a ferromagnetic material on a portion of the housing;

FIG. 11A depicts a cross-sectional view of yet another pair of beads for use with the sphincter device of FIG. 6, each bead including a magnet surrounded by a filling material, and a machine configured to produce a magnetic field oriented within range of the beads, with the machine in a non-activated state and the magnets in a default position;

FIG. 11B depicts a cross-sectional view of the beads and machine of FIG. 11A, with the machine in an activated state and the magnets in a partially aligned position relative to the magnetic field of the machine;

FIG. 12A depicts a top plan view of a pair of beads that may be incorporated into sphincter device of FIG. 6, including magnets contained within housings that are connected with rigid wires, and a machine capable of applying a magnetic field, with the machine in a non-activated state before applying a magnetic field to the beads;

FIG. 12B depicts a top plan view of the pair of beads of FIG. 12A, with the machine in an activated state applying a magnetic field to the beads;

FIG. 12C depicts a top plan view of the pair of beads of FIG. 12A, with the machine in the non-activated state after applying a magnetic field to the beads;

FIG. 13A depicts a top plan view of another pair of beads that may be incorporated into sphincter device of FIG. 6, including magnets inside housings that are connected with a flexible interconnection, and a machine capable of applying a magnetic field, with the machine in a non-activated state before applying a magnetic field to the beads;

FIG. 13B depicts a top plan view of the pair of beads of FIG. 13A, with the machine in an activated state applying a magnetic field to the beads;

FIG. 13C depicts a top plan view of the pair of beads of FIG. 13A, with the machine in the non-activated state after applying a magnetic field to the beads;

FIG. 14A depicts a top plan view of another example of a sphincter augmentation device including a band and a pair of rotatable magnetic beads, with the sphincter augmentation device in an expanded configuration;

FIG. 14B depicts a top plan view of the sphincter augmentation device of FIG. 14A, with the sphincter augmentation device in a contracted configuration, with pair of magnetic beads approximated;

FIG. 15A depicts a side view of a portion of the sphincter augmentation device of FIG. 14A and a machine capable of producing a magnetic field, with the machine in a non-activated state and the beads in a default position;

FIG. 15B depicts a side view of the portion of the sphincter augmentation device of FIG. 15A, with the machine in an activated state and the beads in an aligned position relative to the magnetic field of the machine;

FIG. 16A depicts a top plan view of yet another example of a sphincter augmentation device including a plurality of bands and a plurality of magnets, with the sphincter augmentation device in an expanded configuration;

FIG. 16B depicts a top plan view of the sphincter augmentation device of FIG. 16A in a contracted configuration;

FIG. 17A depicts a top plan view of the sphincter augmentation device of FIG. 16A in the expanded configuration and a machine capable of producing a magnetic field, with the machine in a non-activated state and the beads in a default position;

FIG. 17B depicts a top plan view of a sphincter augmentation device of FIG. 16A, with the machine in an activated state and the plurality of bands in an aligned position relative to the magnetic field and the plurality of beads in an aligned position relative to the magnetic field;

FIG. 18A depicts a side view of a portion of the sphincter augmentation device of FIG. 16A and a machine capable of producing a magnetic field, with the machine in the non-activated state and the beads in a default position;

FIG. 18B depicts a side view of the portion of the sphincter augmentation device of FIG. 18A, with the machine in the activated state and the beads in an aligned position relative to the magnetic field of the machine;

FIG. 19A depicts a top plan view of a portion of yet another sphincter augmentation device including a plurality of beads linked together with a linking structure having a plurality of swivel joints forming a default shape and a machine capable of producing a magnetic field, with the machine in a non-activated state and the plurality of beads in a default position;

FIG. 19B depicts a top plan view of the portion of the sphincter augmentation device of FIG. 19A, with the machine in the activated state and the linking structure articulating to align the plurality of beads with the magnetic field;

FIG. 20 depicts a top plan view of yet another portion of a sphincter augmentation device including a plurality of magnets and a plurality of linking structures having joints and a loop that surrounds a rod to allow a planned movement;

FIG. 21 depicts a top plan view of another pair of beads for incorporating into the sphincter augmentation device of FIG. 6 including a plurality of magnets and linking structure including a loop, a rod, and a stabilizer bar;

FIG. 22A depicts a cross-sectional isometric view, taken along a coronal plane of a human esophago-gastric junction with a flexible endoscopic device including a bladder in a partially expanded position measuring the internal diameter of an LES;

FIG. 22B depicts a cross-sectional isometric view, taken along a coronal plane of a human esophago-gastric junction with the flexible endoscopic device of FIG. 22A, with the bladder in a fully expanded position measuring the restriction force of the LES;

FIG. 23 depicts a cross-sectional isometric view, taken along a coronal plane of a human esophagus and a portion of a stomach of a patient, with a flexible endoscopic device transorally inserted in the esophagus deploying a magnetic sphincter device while being monitored by a laparoscopic device inserted through an incision in the abdomen;

FIG. 24A depicts an enlarged cross-sectional isometric view, taken along a coronal plane of the body, of a human esophago-gastric junction, with the flexible endoscopic device of FIG. 23 positioned to size and analyze tissue characteristics while being monitored with laparoscopic device of FIG. 23;

FIG. 24B depicts a cross-sectional isometric view, taken along a coronal plane of the body, of a human esophago-gastric junction, with a sharp obturator of the flexible endoscopic device of FIG. 23 disposed obliquely through a sidewall of the esophagus, creating a transverse opening;

FIG. 24C depicts a cross-sectional isometric view, taken along a coronal plane of the body, of a human esophago-gastric junction, with the flexible endoscopic device of FIG. 23 deploying a pull wire around the LES by a grasper;

FIG. 24D depicts a cross-sectional isometric view taken along a coronal plane of the body, of a human esophago-gastric junction, with the flexible endoscopic device of FIG. 23 attaching a hook of the pull wire to a suture loop of a sphincter augmentation device, while being monitored with the laparoscopic device of FIG. 23;

FIG. 24E depicts a cross-sectional isometric view, taken along a coronal plane of the body, of a human esophago-gastric junction, with the sphincter augmentation device of FIG. 24D deployed around the LES;

FIG. 24F depicts a cross-sectional isometric view, taken along a coronal plane of the body, of a human esophago-gastric junction, with clasp features of the sphincter augmentation device coupled together while the sphincter augmentation device is deployed around the LES;

FIG. 24G depicts a cross-sectional isometric view, taken along a coronal plane of the body, of a human esophago-gastric junction, with a flexible grasper inserted through a lumen of the endoscopic device of FIG. 23 to position the sphincter augmentation device to a desired position based on the analysis of an imaging camera that analyzes the tissue to determine the desired position;

FIG. 24H depicts a cross-sectional isometric view, taken along a coronal plane of the body, of a human esophago-gastric junction, with the flexible grasper of FIG. 24G manipulating a suture to close the transverse opening of FIG. 24B; and

FIG. 24I depicts a cross-sectional isometric view, taken along a coronal plane of the body, of a human esophago-gastric junction, with the flexile grasper of FIG. 24G withdrawn from the anatomical passage with the transverse opening of FIG. 24B remaining closed with the suture.

The drawings are not intended to be limiting in any way, and it is contemplated that various embodiments of the technology may be carried out in a variety of other ways, including those not necessarily depicted in the drawings. The accompanying drawings incorporated in and forming a part of the specification illustrate several aspects of the present technology, and together with the description serve to explain the principles of the technology; it being understood, however, that this technology is not limited to the precise arrangements shown.

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

### I. OVERVIEW OF EXAMPLE OF SPHINCTER AUGMENTATION DEVICE

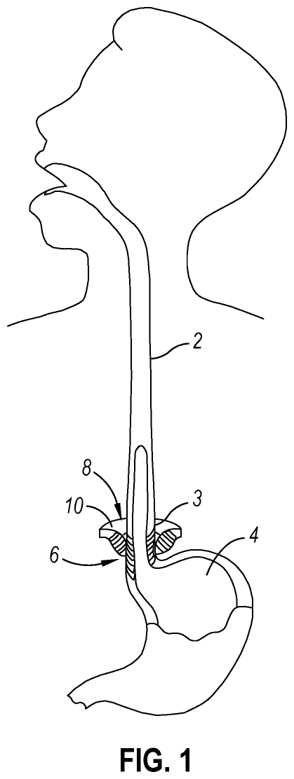

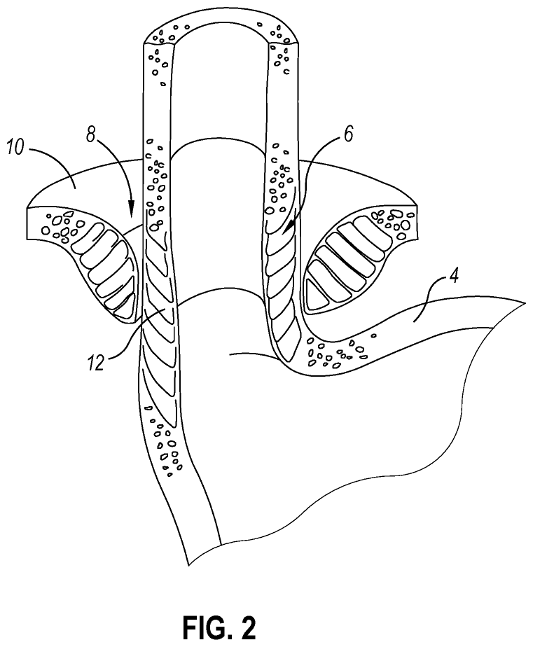

FIGS. 1-2 show selected portions of human anatomy, which includes an esophagus (2) extending from the mouth, through a hiatus (8) defined by a diaphragm (10), and into a stomach (4). Esophagus (2) also includes a distal esophagus (3) and an LES (6). LES (6) is located along distal esophagus (3) adjacent to the junction of esophagus (2) and stomach (4). The portion of LES (6) extending through hiatus (8) is supported by diaphragm (10). When functioning properly, LES (6) is configured to transition between an occluded state and an opened state (as shown in FIG. 2). As best seen in FIG. 2, LES (6) includes a plurality of sling fibers (12). Sling fibers (12) are smooth muscle tissue that may help regulate LES (6) transition between the occluded state and the open state. Hiatus (8) of diaphragm (10) may also help LES (6) transition between the occluded state and the open state.

A healthy LES (6) transitions between the occluded state and the opened state to act as a valve. In other words, a healthy LES (6) may transition from the occluded state to the opened state to allow solids, liquids, and/or gasses to selectively travel between esophagus (2) and stomach (4). For example, a healthy LES (6) may transition from the occluded state to the opened state to permit a bolus of food to travel from esophagus (2) into stomach (4) during peristalsis; or to vent intra-gastric pressure from stomach (4) toward esophagus (2). Additionally, in the occluded state, a healthy LES (6) may prevent digesting food and acidic fluid from exiting stomach (4) back into esophagus (2).

If LES (6) ceases functioning properly by prematurely relaxing, and thereby improperly transitioning esophagus (2) from the occluded state to the opened state, undesirable consequences may occur. Examples of such undesirable consequences may include acidic reflux from stomach (4) into esophagus (2), esophageal damage, inflamed or ulcerated mucosa, hiatal hernias, other GERD symptoms, or other undesirable consequences as will be apparent to one having ordinary skill in the art in view of the teachings herein. Therefore, if an individual has an LES (6) that prematurely relaxes, causing improper transitions from the occluded state to the opened state, it may be desirable to insert an implant around a malfunctioning LES (6) such that the implant and/or LES (6) may properly transition between the occluded state and the opened state.

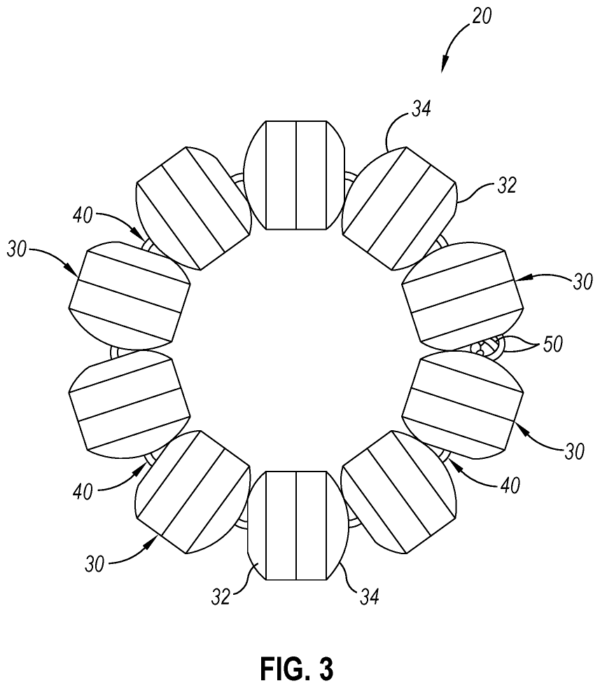

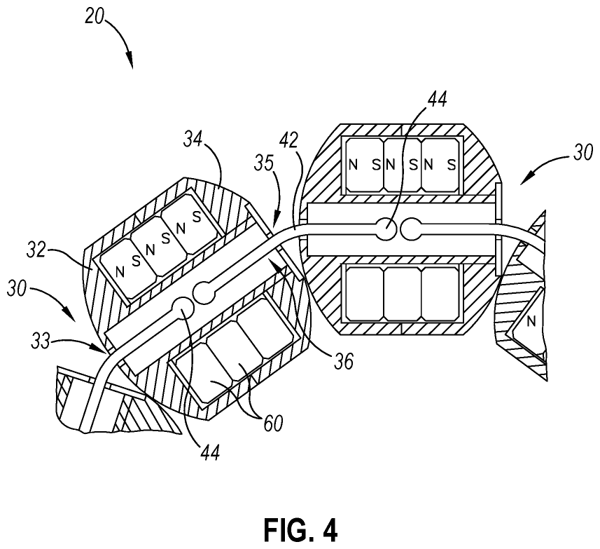

FIGS. 3-5B show an example of a sphincter augmentation device (20) that may be used as an implant around a malfunctioning LES (6) to assist the LES (6) in transitioning between the occluded state and the opened state. Device (20) of this example comprises a plurality of beads (30) that are joined together by a plurality of links (40). Each bead (30) comprises a pair of housings (32, 34) that are securely fastened to each other. By way of example only, housings (32, 34) may be formed of a non-ferrous material (e.g., titanium, plastic, etc.). Each bead (30) further comprises a plurality of annular or toroidal rare-earth permanent magnets (60) that are stacked next to each other within housings (32, 34). In the present example, magnets (60) are completely sealed within beads (30). As best seen in FIG. 4, each bead (30) also defines a chamber (36) that is configured to receive a portion of a respective pair of links (40). Housing (32) defines an opening (33) at one end of chamber (36); while housing (34) defines an opening (35) at the other end of chamber (36).

Each link (40) of the present example comprises a wire (42) that is pre-bent to form an obtuse angle. The free end of each wire (42) terminates in a ball tip (44). Beads (30) are joined together by links (40) such that a first end portion of a link (40) is in one bead (30), a second end portion of the same link (40) is in another bead (30), and an intermediate portion of the same link (40) is positioned between those two beads (30). Chambers (36) of beads (30) are configured to freely receive ball tips (44) and adjacent regions of wires (42); while openings (33, 35) are configured to prevent ball tips (44) from exiting chambers (36). Openings (33, 35) are nevertheless sized to allow wire (42) to slide through openings (33, 35). Thus, links (40) and beads (30) are configured to allow beads (30) to slide along links (40) through a restricted range of motion.

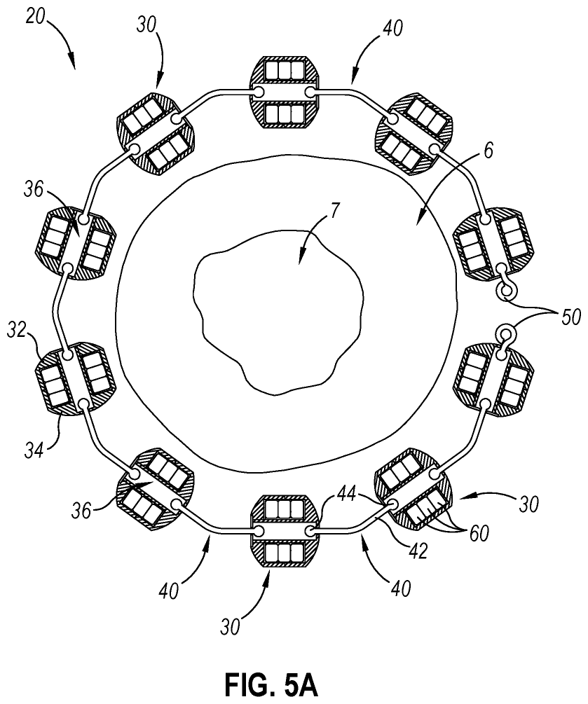

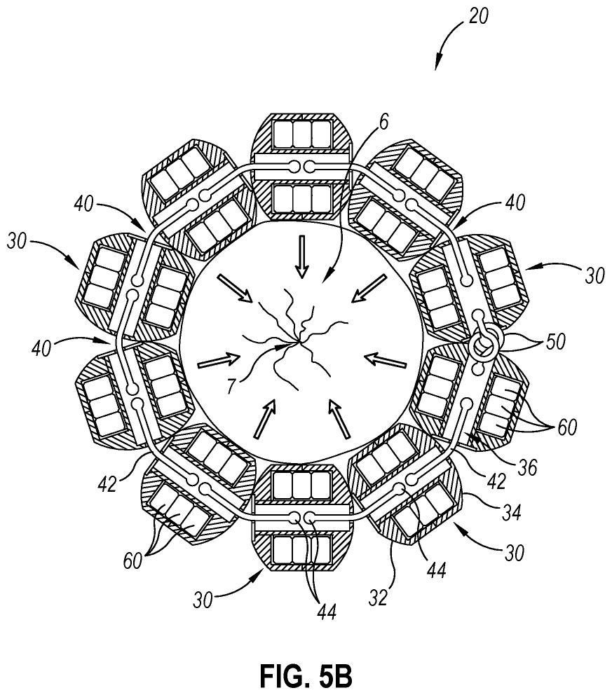

As best seen in FIGS. 5A-5B, two beads (30) have opposing fastener features (50) that allow the ends of device (20) to be coupled together to form a loop. In the present example, fastener features (50) comprise eyelets. In some other versions, fastener features (50) comprise complementary clasp features. As another merely illustrative example, fastener features (50) may be configured and operable in accordance with one or more of the teachings of U.S. Pat. No. 10,405,865, entitled “Method for Assisting a Sphincter,” issued Sep. 10, 2019, the disclosure of which is incorporated by reference herein, in its entirety. Other suitable ways in which the ends of device (20) may be coupled together to form a loop will be apparent to those of ordinary skill in the art in view of the teachings herein. Those of ordinary skill in the art will also recognize that it may be desirable to provide fastener features (50) that can be decoupled if it becomes necessary or otherwise warranted to remove device (20) from the patient.

FIG. 5A shows device (20) in an open, expanded state, with device (20) being positioned about LES (6). At this stage, the opening (7) defined by LES (6) is in a persistently open state (e.g., allowing the patient to undesirably experience GERD and/or other undesirable conditions), warranting the securement of device (20) about the LES (6). FIG. 5B shows device (20) secured about the LES (6), with device (20) in a closed, contracted state. Device (20) is secured closed via fastener features (50). Magnets (60) are oriented within beads (30) such that each bead (30) will be magnetically attracted to the adjacent bead (30) in device (20). In other words, beads (30) are magnetically attracted to each other to magnetically bias device (20) toward the contracted configuration shown in FIG. 5B.

With device (20) secured around the LES (6) and in the contracted configuration, device (20) deforms the LES (6) radially inwardly to substantially close the opening defined by the LES (6). In doing so, device (20) prevents the patient from experiencing GERD and/or other undesirable conditions that may be associated with a persistently open opening (7) at the LES (6). While magnets (60) have a tesla value that is high enough to substantially maintain opening (7) in a closed state to the point of preventing GERD and/or other undesirable conditions that may be associated with a persistently open opening (7), the tesla value of magnets (60) is low enough to allow LES (6) to expand radially outwardly to accommodate passage of a bolus of food, etc. through the opening (7) of LES (6). To accommodate such expansion, beads (30) may simply slide along links (40) to enlarge the effective diameter of device (20) as the bolus passes. After the bolus passes, the magnetic bias of magnets (60) will return device (20) to the contracted state shown in FIG. 5B. Device (20) thus ultimately prevents GERD and/or other undesirable conditions that may be associated with a persistently open opening (7); while still permitting the normal passage of food, etc. from the esophagus (2) to the stomach (4).

In addition to the foregoing, device (20) may be constructed and operable in accordance with at least some of the teachings of U.S. Pat. No. 7,695,427, the disclosure of which is incorporated by reference herein, in its entirety; and/or U.S. Pat. No. 10,405,865, the disclosure of which is incorporated by reference herein, in its entirety.

### II. EXAMPLE OF MRI COMPATIBLE SPHINCTER AUGMENTATION DEVICE

As noted above, a sphincter augmentation device (20) may be implanted within the body around a malfunctioning LES (6) to assist the LES (6) in transitioning between the occluded state and the opened state. In some patients, a procedure involving a patient having a sphincter augmentation device (20) may include placing the patient in the presence of a Magnetic Resonance Imaging (“MRI”) machine that produces a strong magnetic field (M). The MRI magnetic field (M) may urge the magnets (60) to align with the strong MRI magnetic field (M), which may damage sphincter augmentation device (20) in some cases. For instance, in some scenarios where sphincter augmentation device (20) encounters an MRI magnetic field (M), and the MRI magnetic field (M) urges magnets (60) to align with the MRI magnetic field (M), the inability of magnets (60) to move within housings (32, 34) may cause the entirety of each bead (30) to move in such a way that would bring magnets (60) into alignment with the MRI magnetic field (M). Such movement may warp sphincter augmentation device (20) away from an annular configuration, which may cause one or more links (40) to encounter substantial forces that are oriented transversely relative to such one or more links (40). In some such cases, the transversely oriented forces may form kinks or other undesirable bends in links (40). In cases where such kinks or other undesirable bends in links (40) are permanently formed in response to the MRI magnetic field (M), such that the kinks or other undesirable bends in links (40) remain even after sphincter augmentation device (20) no longer encounters the MRI magnetic field (M), sphincter augmentation device (20) may no longer function properly.

In view of the foregoing, it may be desirable to configure the sphincter augmentation device (20) with features that resist alignment with the MRI magnetic field (M) or facilitate the alignment with the MRI magnetic field (M) without damaging the sphincter augmentation device. Examples of such features are described in greater detail below.

### A. Sphincter Augmentation Device with Self-Orienting Magnets

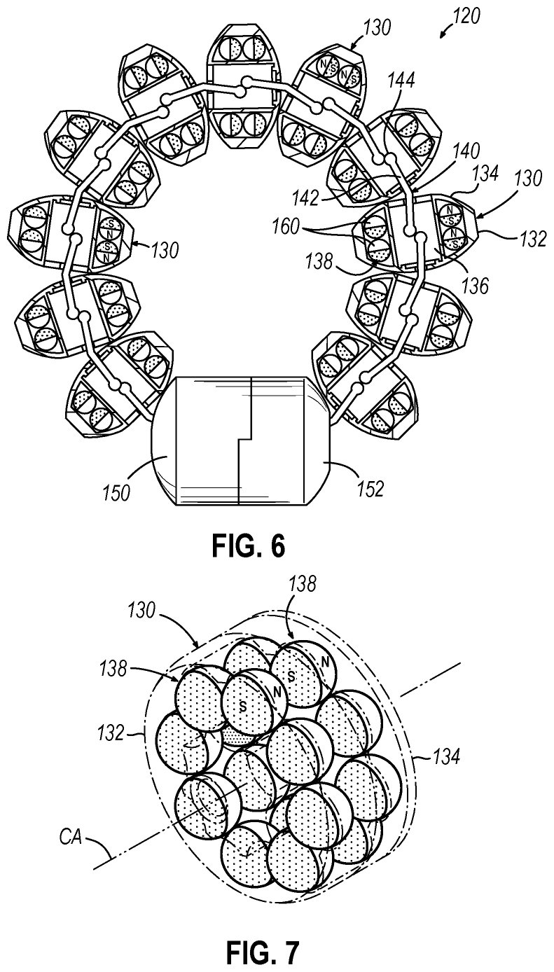

In some instances, it may be desirable to substitute or supplement magnets (60) of sphincter augmentation device (20) with a plurality of spherical magnets (160) that are configured to align with an MRI magnetic field (M), without damaging the sphincter augmentation device (20). FIG. 6 shows an example of another sphincter augmentation device (120) including a plurality of magnets (160) that may be used as an implant around a malfunctioning LES (6) to assist the LES (6) in maintaining an occluded state that will not be damaged by MRI magnetic field (M). Sphincter augmentation device (120) is similar in structure and function to sphincter augmentation device (20) described above except as otherwise described below.

Similar to sphincter augmentation device (20) described above, device (120) of this example comprises a plurality of beads (130) that are joined together by a plurality of links (140). Similar to sphincter augmentation device (20), each bead (130) comprises a housing assembly (132, 134), similar to housings (32, 34) of sphincter augmentation device (20), that is securely fastened together. By way of example only, housing assembly (132, 134) may be formed of a non-ferrous material (e.g., titanium, plastic, etc.). Links (140) of the present example are also similar to links (40) of sphincter augmentation device (20), such that each link (140) comprises a wire (142) that is pre-bent to form an obtuse angle. A free end of each wire (142) terminates in a ball tip (144) to retain each wire (142) within each bead (130). Each bead (130) also defines a chamber (136) that is configured to receive a portion of a respective pair of links (140), as described above. Beads (130) may include two joining beads (150, 152) or fastener features (50) that join the plurality of beads (130) to form a loop.

Device (120) differs from sphincter augmentation device (20) in that device (120) includes a plurality of spherical rare-earth magnets (160) that are free to rotate and orient in different directions within the housing assembly (132, 134). Housing assembly (132, 134) includes a housing (134) and a lid (132). Housing assembly (132, 134) differs from housings (34, 32) in that housing assembly (132, 134) comprises a greater portion of the exterior wall than the pair of housings (32, 34) that comprise approximately equal portions of the exterior wall. Housing (132) at a free end of lid (134) is securely fasted to lid (134). A larger housing (132) facilitates installing the spherical magnets (160) within the housing assembly (132, 134).

Spherical magnets (160) are rare-earth permanent magnets arranged in a pair of annular arrays (138), best shown in FIG. 7, positioned in the outer portions of housings (132,134) about a central magnet axis (CA). In the present example, magnets (160) are completely sealed within beads (130).

FIG. 6 best shows sphincter augmentation device (120) in a default state. Sphincter augmentation device (120) is in a contracted configuration, with each magnet (160) acting upon adjacent magnets (160) within each bead (130) and other beads (130) to radially move inwardly in order to urge the opening (7) defined by the LES (6) to a closed state. The default state is defined as the state where magnets (160) are radially aligned either in the contracted or expanded position (without an MRI magnetic field (M) acting on magnets (160)). By being radially aligned, magnets (160) are angularly oriented about their respective axes such that the poles (N, S) of magnets (160) are all aligned along radial axes extending from a central region of the loop defined by sphincter augmentation device (120). During normal operation, in the default state magnets (160) are magnetically attracted to the opposite magnetic poles of adjacent magnets (160); and magnets (160) are free to rotate and align with each other within the housing assembly (132, 134). Once each magnet (160) is aligned with other magnets (160), sphincter augmentation device (120) is magnetically biased to move from a radially expanded state (not shown) to a radially contracted configuration (FIG. 6).

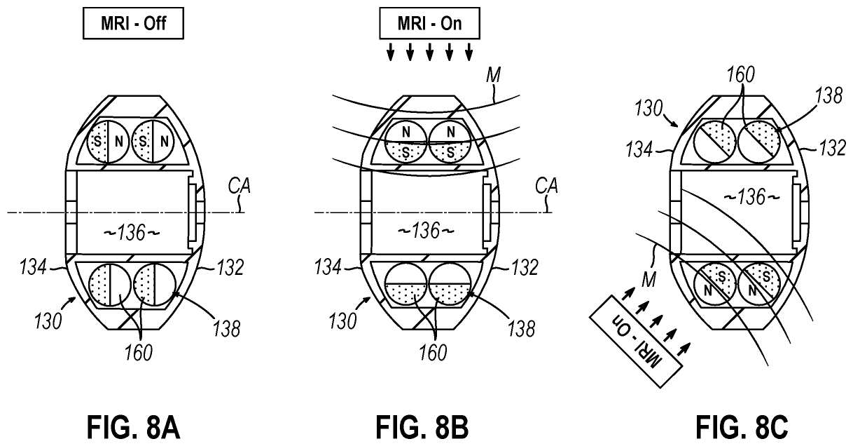

FIG. 8A shows bead (130) proximately located to an MRI machine (MRI). MRI machine (MRI) is positioned parallel to the central axis (CA) of bead (130) that extends through the bead (130) and is in the non-activated state, such that MRI machine (MRI) is not generating an MRI magnetic field (M). With MRI machine (MRI) in the non-activated state, magnets (160) are in the default state with north poles (N) of each magnet (160) oriented facing a first direction relative to central axis (CA) towards an adjacent magnet (not shown).

FIG. 8B shows bead (130) proximately located to the MRI machine (MRI), with the MRI machine (MRI) is positioned parallel to the central axis (CA) of bead (130), and with the MRI machine (MRI) in the activated state such that the MRI machine (MRI) is generating an MRI magnetic field (M). With the MRI machine (MRI) in the activated state, the MRI magnetic field (M) causes each magnet (160) to rotate within housing assembly (132, 134) to align the poles (N, S) of magnets (160) with the MRI magnetic field (M). By being aligned with the MRI magnetic field, magnets (160) are angularly oriented about their respective axes such that the poles (N, S) of magnets (160) are oriented perpendicularly relative to the direction of the magnetic force generated by the MRI magnetic field. Once magnets (160) are in the aligned state relative to MRI magnetic field (M), north poles (N) of each magnet (160) face away transversely away from the central axis (CA) rather than in a first direction relative to central axis (CA) as in FIG. 8A.

FIG. 8C shows bead (130) proximately located to the MRI machine (MRI), with the MRI machine (MRI) obliquely positioned relative to the central axis (CA) of bead (130), and with the MRI machine (MRI) in the activated state such that the MRI machine (MRI) is generating an MRI magnetic field (M). As noted above with respect to FIG. 8B, the MRI magnetic field (M) causes each magnet (160) to rotate within housing assembly (132, 134) to align the poles (N, S) of magnets (160) with the MRI magnetic field (M). However, since the position of the MRI magnetic field (M) is different in FIG. 8C from the position of the MRI magnetic field (M) in FIG. 8B, the angular positions of magnets (160) within housing assembly (132, 134) is correspondingly different in FIG. 8C from the angular positions of magnets (160) within housing assembly (132, 134) in FIG. 8B.

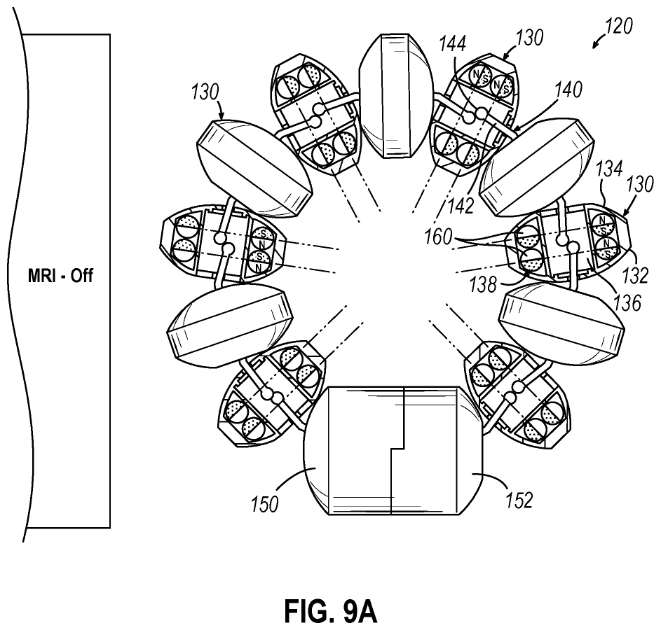

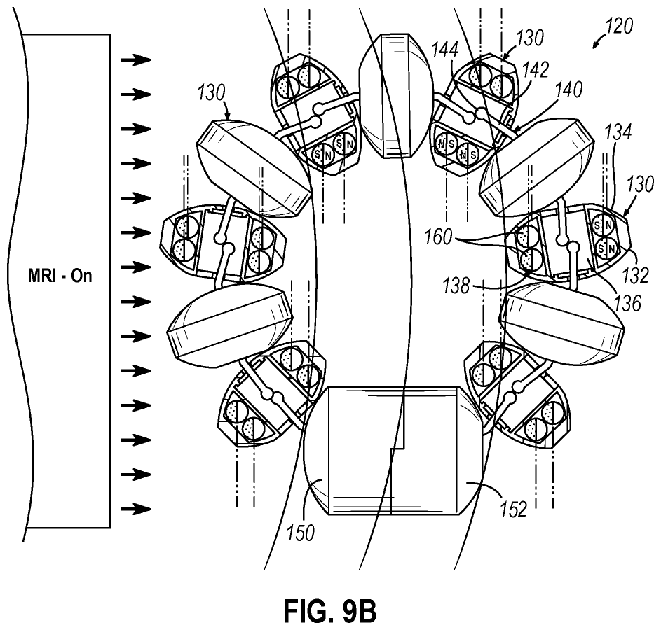

FIG. 9A shows sphincter augmentation device (120) in the default state, and in the contracted configuration, with the magnets (160) acting upon each other with the magnetic poles (N, S) radially aligned with each other and proximately located to an MRI machine (MRI). The MRI machine (MRI) is in the non-activated state, such that the MRI machine (MRI) is not generating an MRI magnetic field (M). With the MRI machine (MRI) in the non-activated state, magnets (160) are in the default angular orientation within housing assemblies (132, 134), with magnetic poles (N, S) of each magnet (160) oriented within their housing assemblies (132, 134) to align with the magnets (160) within bead (130); and to radially align with magnets (160) located in adjacent beads (130). When sphincter augmentation device (120) transitions between the contracted configuration (FIG. 9A) and an expanded configuration (similar to what is shown in FIG. 5A while the MRI machine (MRI) is in the non-activated state, links (140) may generally tend to only encounter tensional forces, such that links (140) may encounter little to no transversely oriented forces (i.e., forces oriented transversely relatively to each link (140)) during normal operation of sphincter augmentation device (120) when MRI machine (MRI) is in the non-activated state, links (140).

FIG. 9B shows sphincter augmentation device (120) in an aligned state, and contracted configuration, with the magnets (160) being acted upon by the MRI machine (MRI). The MRI machine (MRI) is in the activated state and generating an MRI magnetic field (M). With the MRI machine (MRI) in the activated state, the MRI magnetic field (M) causes each magnet (160) to rotate within housing assembly (132, 134) to align the poles (N, S) of magnets (160) with the MRI magnetic field (M) as noted above with respect to FIGS. 8B and 8C. It should be noted that magnets (160) move within housing assemblies (132, 134) without moving beads (130) and without moving or distorting links (140). Thus, due to the spherical configuration of magnets (160), and due to the freedom of movement of magnets (160) within housing assemblies (132, 134), sphincter augmentation device (120) is configured to accommodate alignment of magnets (160) with the MRI magnetic field (M) without causing damage to links (140) or other components of sphincter augmentation device (120).

### B. Sphincter Augmentation Device with Magnetic Field Damping

In some instances, it may be desirable to substitute or supplement portions of a sphincter augmentation device (20) with a bead (230) that includes dissimilar materials, including a ferromagnetic material that is configured to damp an MRI magnetic field (M) and thereby shield against or control the effects of the MRI magnetic field (M), so that the magnets and housing do not align with an MRI magnetic field (M).

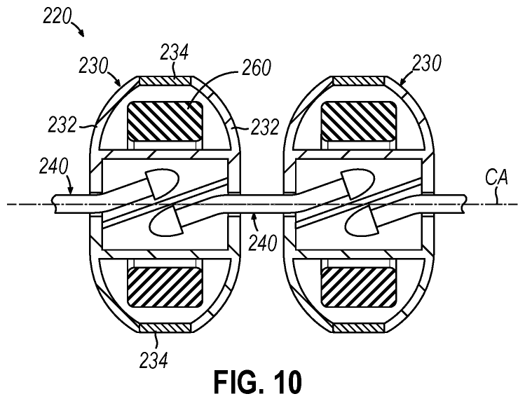

FIG. 10 shows a portion of a sphincter augmentation device (220) including a pair of beads (230) coupled together with a link (240). Each bead (230) includes a magnet (260) contained within a housing assembly (232, 234). Beads (230), links (240), and magnets (260) are similar in structure and function to beads (30), links (40), and magnets (60) of sphincter augmentation device (20) except as otherwise noted below. Housing assembly (232, 234) of beads (230) differs from housings (32, 34) of beads (30). Each housing assembly (232, 234) extends along a respective central axis (CA). Each housing assembly (232, 234) includes a pair of end caps (232) and a middle portion (234) positioned between the pair of end caps (232). End caps (232) are fixedly secured to middle portion (234). Each end cap (232) has a convex shaped exterior surface and middle portion (234) has a cylindrical shaped exterior surface. End caps (232) are formed of titanium or some other non-ferromagnetic material, so the magnetic field crated by magnet (260) in bead (230) attracts an adjacent magnet (260) positioned within an adjacent bead (230) with enough force to urge sphincter augmentation device (220) from an expanded configuration toward a contracted configuration. Magnets (260) may be annular, toroidal, spherical, or otherwise shaped. In some versions, magnets (260) are rare-earth magnets. In the present version, there is one annular magnet (260) housed within each bead (230), though other versions may have more than one magnet (260) in each bead (230).

In the present example, middle portion (234) is formed of one or more ferromagnetic materials such as iron, nickel, cobalt, or a rare earth metal. Additionally, middle portion (234) may include a non-ferromagnetic material coated with a ferromagnetic coating. Alternatively, middle portion (234) may include a ferromagnetic material in some other fashion. By being formed of a ferromagnetic material, or by otherwise including a ferromagnetic material, middle portion (234) may provide a damping effect with respect to an MRI magnetic field (M). Middle portion (234) is thus configured to resist the magnetic attraction from external magnetic fields such as an MRI magnetic field (M), so that sphincter augmentation device (20) that incorporates beads (230) does not distort in such a way as to kink or otherwise damage links (240) in response to an MRI magnetic field (M). While the ferromagnetic properties of middle portion (234) may provide a damping effect on an MRI magnetic field (M), the non-ferromagnetic properties of end caps (232) may allow magnets (260) to cause attraction between adjacent beads (230), such that sphincter augmentation device (220) may still assist a sphincter like the LES (6) to achieve a contracted state. In other words, the ferromagnetic properties of middle portion (234) may not have an adverse effect on magnetic attraction between adjacent beads (230) even if middle portion (234) of each bead (230) damps an MRI magnetic field (M).

### C. Sphincter Augmentation Device with Dampening Material Filled Beads

In some instances, it may be desirable to substitute or supplement portions of a sphincter augmentation device (20) with a bead (330) with a dampening material (350) that surrounds magnets (360) and prevents magnets (360) from making quick, abrupt, or large movements in response to an externally applied strong magnetic field such as an MRI magnetic field (M) from an MRI machine (MRI).

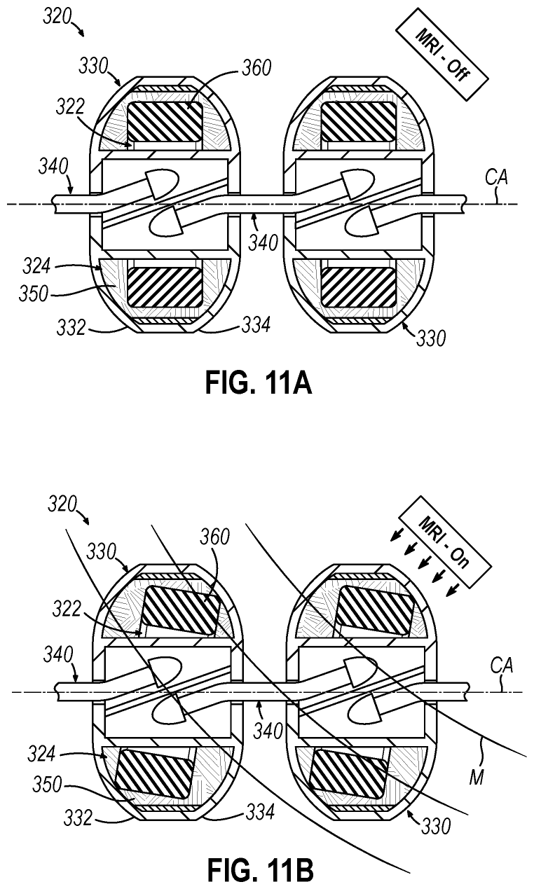

FIG. 11A shows a portion of a sphincter augmentation device (320) that includes a pair of beads (330) and links (340). Each bead (330) includes a magnet (360), housing assemblies (322, 324), and a dampening material (350). Housing assemblies (332,334), links (340), and magnets (360) are similar in structure and function to pair of housings (32,34), links (40), and magnets (60) of sphincter augmentation device (20) except as otherwise noted below. Each housing assembly (332, 334) extends along a central axis (CA). Links (340) are configured to slidably couple adjacent beads (330) together. Housing assembly (332, 334) is formed of a suitable material that has sufficient rigidity, such as titanium. Magnets (360) may be annular, toroidal, or otherwise shaped. In some versions, magnets (360) are rare-earth magnets. In the present version, there is one magnet (360) housed within each bead (330), but any suitable number of magnets may be contained within each housing assembly (332, 334).

In FIG. 11A, beads (330) are shown in close proximity to an MRI machine (MRI), with the MRI machine (MRI) in a non-activated state, such that each magnet (360) is in a default position where each magnet (360) is not being acted upon by MRI magnetic field (M). In the default position, magnet (360) is positioned with housing assembly (332, 334) with a central opening (322) of magnet (360) aligned with the central axis (CA). Dampening material (350) fills a portion of the bead interior (324), such that each magnet (360) is surrounded by dampening material (350). In versions where each bead (330) includes more than one magnet (360), dampening material (350) may be interposed between each magnet (360) within each bead (330). Dampening material (350) may comprise an elastically deformable material (e.g., polypropylene foam, etc.).

In some versions, the dampening material (350) maybe be applied to the housing assembly (332,334) by an injection molding process that produces a molded scaffold or casing constructed of a malleable or otherwise deformable material such as polypropylene. This malleable material is configured to provide a deformable cushion when external fields such as an MRI magnetic field (M) are applied. In some versions, dampening material (350) may be formed of a low-density polypropylene, similar in form to a clay, that is configured to provide a damper to rapid vibrational movements of magnets (360) within housing assemblies (332, 334).

FIG. 11B shows the pair of beads (330) being acted upon by the MRI magnetic field (M) with the MRI machine (MRI) in an activated state. The MRI magnetic field (M) acts upon magnets (360), urging the magnetic poles (N, S) to align with the MRI magnetic field (M). This magnetic influence on magnets (360) causes each magnet (360) to move within its respective housing assembly (332, 334). Dampening material (350) deforms to accommodate such movement of each magnet (360) relative to the corresponding housing assembly (332, 334). While dampening material (350) deforms to accommodate such movement of each magnet (360) relative to the corresponding housing assembly (332, 334), dampening material (350) provides mechanical dampening of such movement, such that dampening material (350) prevent magnets (360) from making quick, abrupt or large movements in response to the MRI magnetic field (M). Dampening material (350) thus prevents sudden movements of beads (330) that might otherwise cause kinking or other damage to links (340) in response to the MRI magnetic field (M).

When the MRI machine (MRI) transitions back to the non-activated state, dampening material (350) may resiliently urge magnets (360) back to the default position within their respective housing assembly (332, 334). In other words, after the MRI magnetic field (M) is removed, beads (330) may return to the state shown in FIG. 11A. In versions where dampening material (350) is not resilient, magnetic attraction between magnets (360) of adjacent beads (330) may cause magnets (360) to return to the default position within their respective housing assembly (332, 334) when the MRI machine (MRI) transitions back to the non-activated state. In such versions, dampening material (350) may accommodate such magnetically induced movement of magnets (360) back from the deflected position shown in FIG. 11B to the default position shown in FIG. 11A.

### D. Sphincter Augmentation Device with MRI Compatible Linking Structure

In some instances, it may be desirable to substitute or supplement portions of a sphincter augmentation device (20) with a version of link (40) including variations of wires (42) that allow twisting and rotating without damaging the link (40) when sphincter augmentation device (20) is exposed to a strong externally applied magnetic field such as an MRI magnetic field (M) from an MRI machine (MRI).

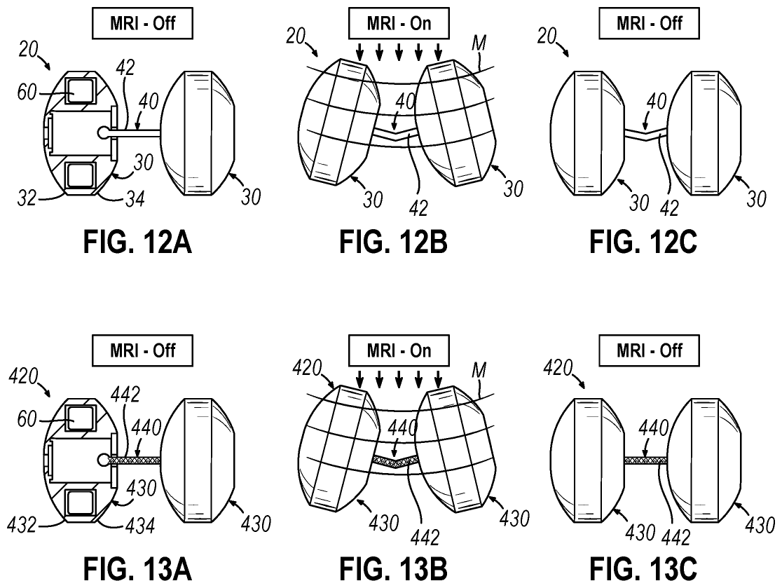

FIG. 12A shows a pair of beads (30) and a link (40) of sphincter augmentation device (20) in close proximity to an MRI machine (MRI), with beads (30) in a default position, not being acted upon by MRI magnetic field (M). MRI machine (MRI) is in a de-activated state.

FIG. 13A shows a portion of a sphincter augmentation device (420) including a pair of beads (430) and a link (440) in close proximity to an MRI machine (MRI), beads (430) in a default position, not being acted upon by MRI magnetic field (M). Each bead (430) includes a magnet (460) disposed in a housing assembly (432, 434), with links (440) joining beads (430) together. Beads (430), link (440), and magnets (460) are similar in structure and function to beads (30), link (40), and magnets (60) of sphincter augmentation device (20) except as otherwise noted below. Link (440) include interconnects (442) that allow housing assemblies (432, 434) to freely adjust to the MRI magnetic field (M), with interconnects (442) accommodating the twist of some of the beads (430) with respect to each other and other kinds of movements of beads (430) with respect to each other. Interconnects (442) are configured to transform shape when MRI magnetic field (M) is applied; but return to the original state after the MRI magnetic field (M) is no longer applied.

Links (440) include interconnects (442) that are resilient and allow beads (430) to rotate more freely when MRI magnetic field (M) is applied. Interconnects (442) are constructed of a flexible material capable of being distorted but capable of regaining shape such as a liquid crystal polymer weave, or a nylon weave. In some versions, interconnects (442) are formed of one or more single strands of a polymeric material, which may be woven or non-woven. Also in some versions, interconnects (442) are resiliently biased to assume the configuration shown in FIG. 13A. In some other versions, interconnects (442) are not resilient.

FIG. 12B shows beads (30) and link (40) of sphincter augmentation device (20) in close proximity to an activated MRI machine (MRI), such that beads (30) are being acted upon by an MRI magnetic field (M). Magnets (60) align with the MRI magnetic field (M), thereby distorting the wire (42) of the link (40), thereby creating a permanent kink or bend (43) in wire (42).

FIG. 13B shows beads (430) and link (440) in close proximity to an activated MRI machine (MRI), such that beads (430) are being acted upon by an MRI magnetic field (M). Magnets (460) align with the MRI magnetic field (M), thereby distorting the interconnect (442) of the link (440), thereby creating a non-permanent bend (443) in interconnect (442). In other words, due to the different material properties of link (440) as compared to link (40), link (440) is configured to accommodate greater distortion than link (40) without permanently deforming.

FIG. 12C shows beads (30) and link (40) after being acted upon by the MRI magnetic field (M). Since the MRI magnetic field (M) has been removed at this stage, magnets (60) are no longer aligned with the MRI magnetic field (M). However, permanent bend (43) remains, such that wire (42) is in a damaged state.

FIG. 13C beads (430) and link (440) after being acted upon by the MRI magnetic field (M). Since the MRI magnetic field (M) has been removed at this stage, magnets (460) are no longer aligned with the MRI magnetic field (M), but non-permanent bend (443) interconnect (442) no longer remains in interconnect (442). Thus, interconnect (442) is not in a damaged state. Thus, by using a polymeric material (e.g., woven liquid crystal polymer, woven nylon, one or more liquid crystal polymer strands, one or more nylon strands, etc.) instead of a single metallic wire (e.g., nitinol), each interconnect (442) is configured to accommodate substantial distortions caused by exposure to MRI magnetic fields (M) without encountering damage.

### E. Sphincter Augmentation Device with Pair of Rotatable Magnetic Beads and Continuous Band

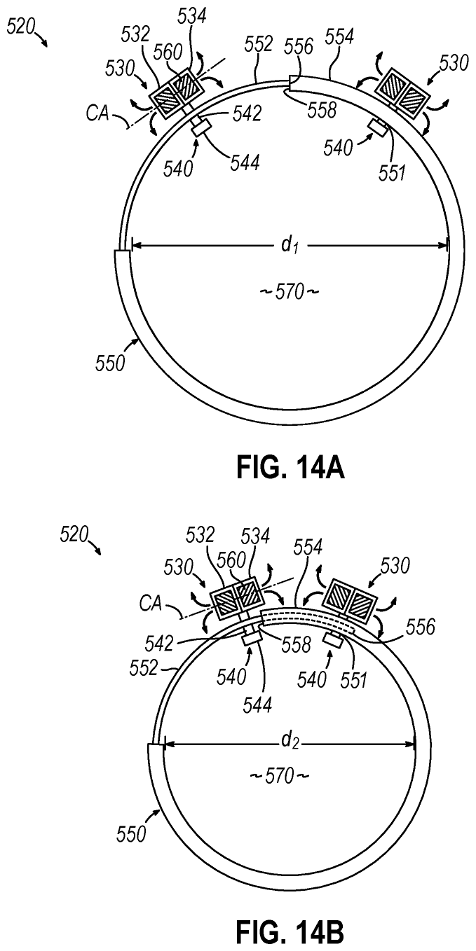

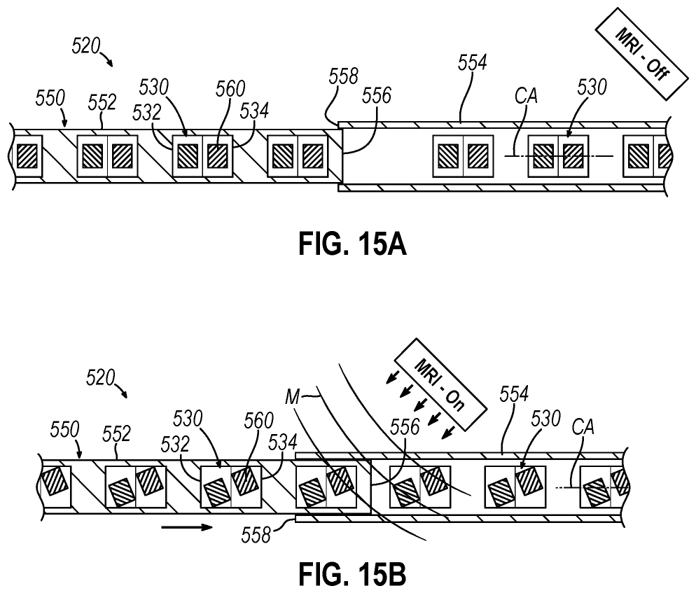

In some instances, it may be desirable to provide a variation of sphincter augmentation device (20) that includes a continuous band and magnets that are configured to align with an MRI magnetic field (M), without damaging the continuous band or other components of the variation of sphincter augmentation device (20). To that end, FIGS. 14A-15B show an example of another sphincter augmentation device (520) that maybe used as an implant around a malfunctioning LES (6) to assist the LES (6) in achieving the occluded state. Sphincter augmentation device (520) is configured such that sphincter augmentation device (520) will not be damaged by the MRI magnetic field (M). Sphincter augmentation device (520) is similar in structure and function to sphincter augmentation device (20) described above except as otherwise described below.

Similar to sphincter augmentation device (20) described above, sphincter augmentation device (520) of this example comprises a pair of beads (530, 531) that are joined together by respective linkage assemblies (540) and a continuous band (550) to form an annular loop that is configured to be installed around a malfunctioning LES (6). Similar to sphincter augmentation device (20), each bead (530, 531) of device (520) may include a housing assembly (532, 534), similar to housings (32, 34), that is securely fastened together. Each housing assembly (532, 534) has one or more magnets (560) positioned within housing assembly (532, 534). By way of example only, housing assembly (532, 534) may be formed of a non-ferrous material (e.g., titanium, plastic, etc.). Magnets (560) may be similar in construction and function to any of the aforementioned magnets (160, 260, 360, 460).

Each linkage assembly (540) comprises a pin (542) and a tab (544). Each pin (542) is fixedly secured to a corresponding housing assembly (532, 534). In some versions, each pin (542) is also fixedly secured to magnet (560) within housing assembly (532, 534). Pins (542) are also slidably disposed in continuous band (550) as described in greater detail below. Each tab (544) extends transversely relative to the corresponding pin (542) and thereby retains each bead (530, 531) relative to continuous band (550). Continuous band (550) and linkage assemblies (540) thus couple beads (530, 531) together instead of utilizing links (40). Beads (530, 531) are arranged such that pins (542) extend radially relative to a central axis (CA) defined by sphincter augmentation device (520).

Continuous band (550) includes a first portion (552) having a first end (556); and a second portion (554) having a second end (558). First portion (552) is configured to be slidably fastened to second portion (554) so that continuous band (550) forms an annular loop. Second portion (554) is tubular in structure and is sized larger than first portion (552) so that first portion (552) may slide within second portion (554). In some versions, second end (558) may be crimped or otherwise reduced in size to hold an enlarged first end (556) so that once installed, first portion (552) cannot be removed from the second portion (554). In some such versions, continuous band (550) may be cut in order to remove sphincter augmentation device (20) from the patient, if the need ever arises. Continuous band (550) defines apertures that are configured to receive pins (542) of linkage assemblies (540) while providing some freedom of movement of beads (530, 531) relative to continuous band (550). In some versions, apertures (551) are circular openings, such that pins (542) may rotate within apertures (551) but not slide along apertures (551). In some other versions, apertures (551) are elongate slots, such that pins (542) may slide along apertures (551) in addition to being able to rotate within apertures (551). Apertures (551) may also be configured to allow pins (542) to pivot relative to continuous band (550) about axes that are transverse relative to pins (542). Alternatively, apertures (551) may have any other suitable configuration. In the present example, a first bead (530) is positioned on the first portion (552) of the continuous band (550) and a second bead (531) is positioned on the second portion (554) of the continuous band (550).

FIG. 14A shows sphincter augmentation device (520) with the first portion (552) of continuous band (550) slidably joined to the second portion (554), forming a band opening (570) in a default state, not being acted on by an MRI magnetic field (M). It should be understood that sphincter augmentation device (520) may be initially provided in a non-loop configuration, thereby enabling the clinician to position sphincter augmentation device (520) about the LES (6). Once sphincter augmentation device (520) has been positioned about the LES (6), the clinician may engage portions (552, 554) of continuous band (550) together to form the loop around the LES (6). Alternatively, sphincter augmentation device (520) may have a clasp feature, etc. In the state shown in FIG. 14A, band opening (570) has a first diameter (d1) and is in an expanded configuration. Magnets (560) are magnetically attracted to each other due to the orientation of opposing poles (N, S) of magnets (560). In some cases, one or both of magnets (560) rotate about pins (542) to position opposing poles (N, S) of magnets (560) toward each other.

FIG. 14B shows sphincter augmentation device (520) after being transitioned to a second diameter (d2) from the first diameter (d1) in response to the magnetic attraction of magnet (560) positioned in first bead (530) being magnetically attracted to magnet (560) positioned in second bead (531). In other words, magnetic bias between beads (530, 531) transitions sphincter augmentation device (520) from the expanded configuration to the contracted configuration. First end (556) of first portion (553) slides deeper into the second end (558) of second portion (554) when sphincter augmentation device (520) is in the contracted configuration.

With sphincter augmentation device (520) secured around the LES (6) and in the contracted configuration, sphincter augmentation device (520) deforms the LES (6) radially inwardly to substantially close the opening (7) defined by the LES (6). In doing so, device (520) prevents the patient from experiencing GERD and/or other undesirable conditions that may be associated with a persistently open opening (7) at the LES (6). While magnets (560) have a tesla value that is high enough to substantially maintain opening (7) in a closed state to the point of preventing GERD and/or other undesirable conditions that may be associated with a persistently open opening (7), the tesla value of magnets (560) is low enough to allow LES (6) to expand radially outwardly to accommodate passage of a bolus of food, etc. through opening (7) of LES (6). To accommodate such expansion, first end (556) of first portion (553) slides relative to second end (558) of second portion (554) to enlarge the effective diameter of sphincter augmentation device (520) as the bolus passes. After the bolus passes, the magnetic bias of magnets (560) will return sphincter augmentation device (520) to the contracted state shown in FIG. 14B. Device (20) thus ultimately prevents GERD and/or other undesirable conditions that may be associated with a persistently open opening (7); while still permitting the normal passage of food, etc. from the esophagus (2) to the stomach (4).

FIG. 15A shows a portion of continuous band (550) with the first portion (552) slidably joined to the second portion (554) forming the band opening (570) (see FIG. 14A) in a default state, proximately located relative to an MRI machine (MRI) in the non-activated state, such that the MRI machine (MRI) is not producing an MRI magnetic field (M). In this state, beads (530, 531) are angularly oriented about the axes defined by pins (542) such that opposing poles (N, S) of magnets (560) face each other.

FIG. 15B shows the same components shown in FIG. 15A, but with the MRI machine (MRI) in an activated state such that the MRI machine is generating an MRI magnetic field (M). In response to this MRI magnetic field (M), magnets (560) are magnetically urged to align with the MRI magnetic field (M). Sphincter augmentation device (520) accommodates such alignment by allowing beads (530, 531) to rotate relative to continuous band (550) about the axes defined by pins (542). In some cases, pins (542) slide along apertures (551) to further accommodate alignment of magnets (560) with the MRI magnetic field (M). In addition, or in the alternative, pins (542) may pivot about pivot axes that are perpendicular to the axes defined by pins (542), to further accommodate alignment of magnets (560) with the MRI magnetic field (M).

With sphincter augmentation device (520) accommodating alignment by allowing beads (530, 531) to rotate, slide, and/or pivot relative to continuous band (550) in response to the presence of an MRI magnetic field (M), sphincter augmentation device (520) may tolerate the presence of the MRI magnetic field (M) without causing any damage to sphincter augmentation device (520). Once the MRI magnetic field (M) is removed (e.g., when the MRI machine (MRI) is deactivated), sphincter augmentation device (520) may readily return to the state shown in FIGS. 14B and 15A. For instance, once the MRI magnetic field (M) is removed, beads (530, 531) may again rotate, slide, and/or pivot relative to continuous band (550) to allow beads (530, 531) to achieve a relative positioning where opposing poles (N, S) of magnets (560) face each other.

### F. Sphincter Augmentation Device with Plurality of Rotatable Magnetic Beads and Segmented Band

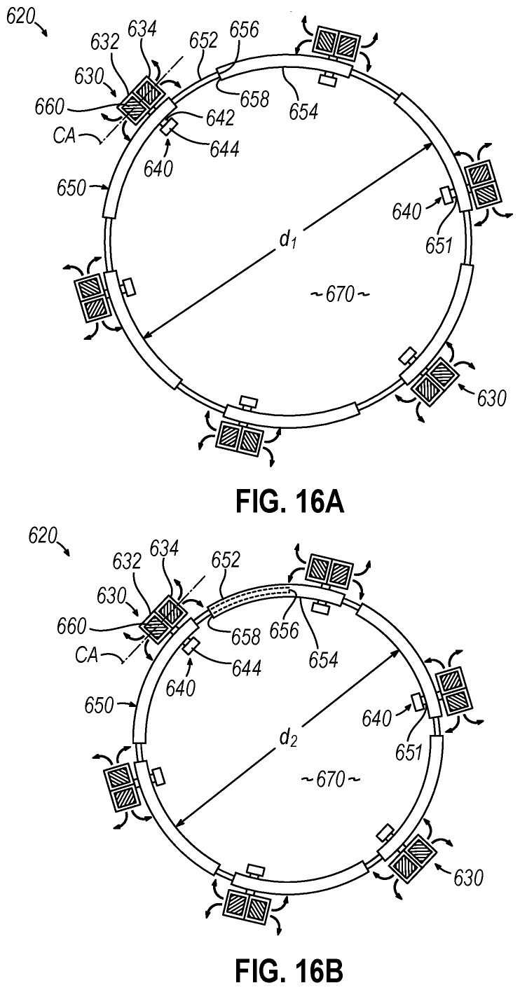

FIGS. 16A-18B show an example of another sphincter augmentation device (620) that may be used as an implant around a malfunctioning LES (6), where sphincter augmentation device (620) will not be damaged by an MRI magnetic field (M). Sphincter augmentation device (620) is similar in structure and function to sphincter augmentation device (520) described above except as otherwise described below.

Similar to sphincter augmentation device (520) described above, sphincter augmentation device (620) of this example comprises a plurality of beads (630) that are joined together by respective linkage assemblies (640) and band segments (650) to form an annular loop that is configured to be installed around a malfunctioning LES (6). Sphincter augmentation device (620) differs from sphincter augmentation device (520) in that sphincter augmentation device (620) has a plurality of beads (630) and a plurality of band segments (650) rather than a pair of beads (530, 531) and a single continuous band (530). Each housing assembly (632, 634) has one or more magnets (660) positioned within housing assembly (632, 634). By way of example only, housing assembly (632, 634) may be formed of a non-ferrous material (e.g., titanium, plastic, etc.). Magnets (660) may be similar in construction and function to any of the aforementioned magnets (160, 260, 360, 460, 560).

Each linkage assembly (640) comprises a pin (642) and a tab (644). Each pin (642) is fixedly secured to a corresponding housing assembly (632, 634). In some versions, each pin (642) is also fixedly secured to magnet (660) within housing assembly (632, 534). Pins (642) are also slidably disposed in respective band segments (650) as described in greater detail below. Each tab (644) extends transversely relative to the corresponding pin (642) and thereby retains each bead (630) relative to the corresponding band segment (650). Band segments (650) and linkage assemblies (640) thus couple beads (630) together instead of utilizing links (40). Beads (630) are arranged such that pins (642) extend radially relative to a central axis (CA) defined by sphincter augmentation device (620).

Each band segment (650) includes a first portion (652) having a first end (656) and a second portion (654) having a second end (658). Ends (656, 658) of adjacent band segments (650) are linked together similar to portions (552, 554) of sphincter augmentation device (520), with the first portions (652) slidably secured within the corresponding second portions (654). Sphincter augmentation device (620) is capable of transitioning between an expanded and a contracted configuration, as described above. Each band segment (650) includes one corresponding bead (630). Each magnet (660) is configured to be magnetically biased toward adjacent magnets (660) to magnetically urge sphincter augmentation device (620) from the expanded configuration to the contracted configuration.

FIG. 16A shows sphincter augmentation device (620) with band segments (650) slidably joined together to form a band opening (670), in a default state, not being acted on by an MRI magnetic field (M). It should be understood that sphincter augmentation device (620) may be initially provided in a non-loop configuration, thereby enabling the clinician to position sphincter augmentation device (620) about the LES (6). Once sphincter augmentation device (620) has been positioned about the LES (6), the clinician may engage two band segments (650) together to form the loop around the LES (6). Alternatively, sphincter augmentation device (620) may have a clasp feature, etc. In the state shown in FIG. 16A, band opening (670) has a first diameter (d1) and is in an expanded configuration. Magnets (660) are magnetically attracted to each other due to the orientation of opposing poles (N, S) of magnets (660). In some cases, at least some of magnets (660) rotate about pins (642) to position opposing poles (N, S) of magnets (660) toward each other.

FIG. 16B shows sphincter augmentation device (620) after being transitioned from the first diameter (d1) to a second diameter (d2) due to the magnetic attraction of magnets (660) to adjacent magnets (660). In other words, magnetic bias between magnets (660) transitions sphincter augmentation device (620) from the expanded configuration to the contracted configuration. Each first end (656) of each first portion (652) slides deeper into the adjacent second end (658) of the adjacent second portion (654) until the contracted configuration is achieved. Thus, magnets (660) magnetically urge adjacent band segments (650) to slide toward each other to transition sphincter augmentation device (620) from the expanded state to the contracted state.

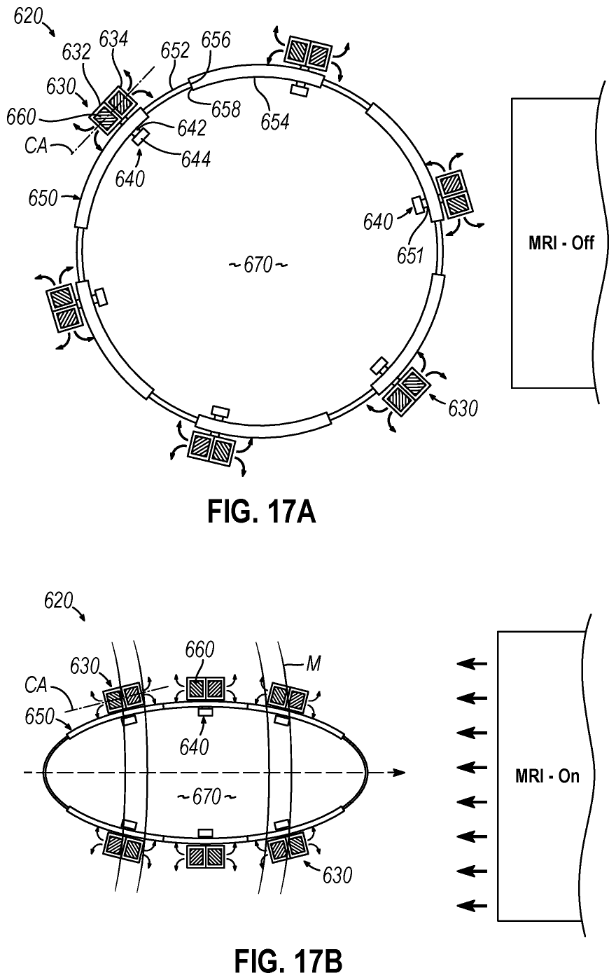

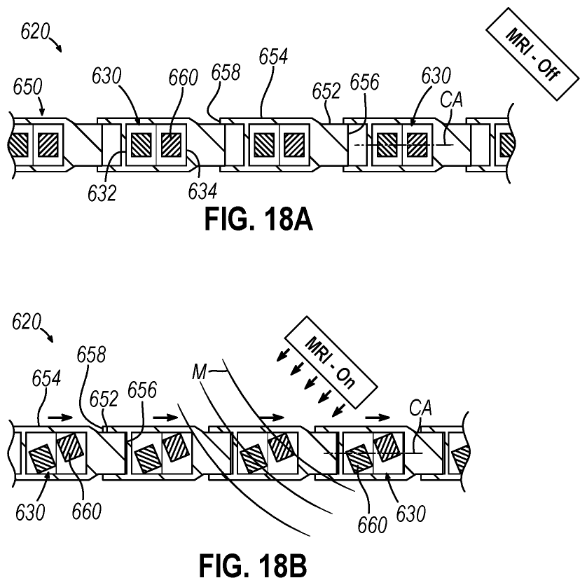

FIGS. 17A and 18A show sphincter augmentation device (620) in the default position, and in the contracted configuration. Device (620) is proximately located relative to an MRI machine (MRI), with the MRI machine (MRI) in a non-activated state such that the MRI machine (MRI) is not producing an MRI magnetic field (M). Band opening (670) is located tangent to the face the MRI machine (MRI) that produces the MRI magnetic field (M). In this state, beads (630) are angularly oriented about the axes defined by pins (642) such that opposing poles (N, S) of adjacent magnets (660) face each other. The magnetic bias of magnets (660) also urges urge adjacent band segments (650) to slide toward each other to thereby achieve the contracted state.

FIGS. 17B and 18B show the same components shown in FIGS. 17A and 18A, respectively, but with the MRI machine (MRI) in an activated state such that the MRI machine is generating an MRI magnetic field (M). In response to this MRI magnetic field (M), magnets (660) are magnetically urged to align with the MRI magnetic field (M). Sphincter augmentation device (620) accommodates such alignment by allowing beads (630) to rotate relative to the corresponding band segments (650) about the axes defined by pins (642). In some cases, pins (642) slide along apertures (651) to further accommodate alignment of magnets (660) with the MRI magnetic field (M). In addition, or in the alternative, pins (642) may pivot about pivot axes that are perpendicular to the axes defined by pins (642), to further accommodate alignment of magnets (660) with the MRI magnetic field (M). As shown in FIG. 17B, alignment of magnets (660) with the MRI magnetic field (M) may also cause band segments (650) to deform, such that sphincter augmentation device (620) forms an oval shaped band opening (670) without necessarily fully constricting the LES (6). In some versions, this oval shaped deformation does not occur in response to an MRI magnetic field (M).

With sphincter augmentation device (620) accommodating alignment by allowing beads (630) to rotate, slide, and/or pivot relative to band segments (650), and by allowing band segments (650) to slide relative to each other (e.g., to collectively form an oval shape, etc.), in response to the presence of an MRI magnetic field (M), sphincter augmentation device (620) may tolerate the presence of the MRI magnetic field (M) without causing any damage to sphincter augmentation device (620). Once the MRI magnetic field (M) is removed (e.g., when the MRI machine (MRI) is deactivated), sphincter augmentation device (620) may readily return to the state shown in FIGS. 16B, 17A, and 18A. For instance, once the MRI magnetic field (M) is removed, beads (630) may again rotate, slide, and/or pivot relative to band segments (650), and/or band segments (650) may slide relative to each other, to allow beads (630) to achieve a relative positioning where opposing poles (N, S) of magnets (660) face each other.

### G. Sphincter Augmentation Device with Pivotable Linking Structure

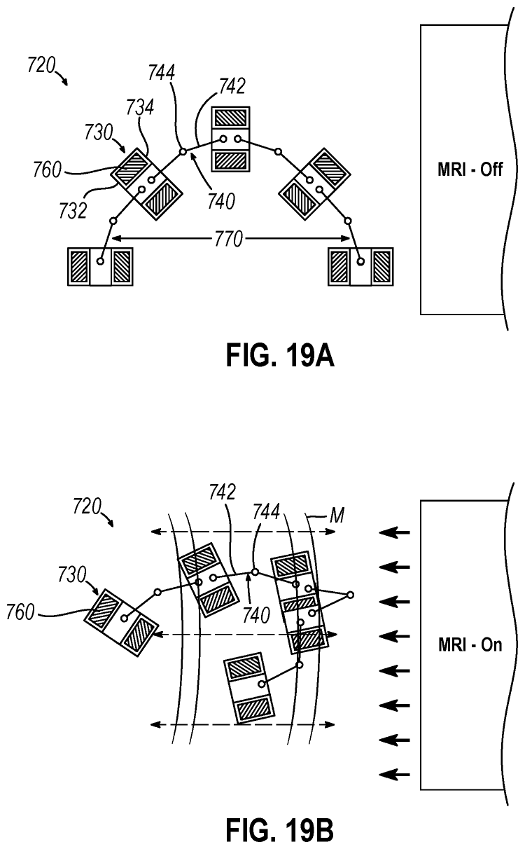

FIGS. 19A-19B show a portion of another exemplary sphincter augmentation device (720) that maybe used as an implant around a malfunctioning LES (6), where sphincter augmentation device (720) will not be damaged by an MRI magnetic field (M). Sphincter augmentation device (720) is similar in structure and function to sphincter augmentation device (520) described above except as otherwise described below.

Similar to sphincter augmentation device (520) described above, sphincter augmentation device (720) of this example comprises a plurality of beads (730) that are joined together by respective linkage assemblies (740) to form an annular loop that is configured to be installed around a malfunctioning LES (6). Each bead (730) of sphincter augmentation device (720) includes a housing assembly (732, 734), similar to housing assemblies (532,534) of sphincter augmentation device (520), that is securely fastened together. Each housing assembly (732, 734) has one or more magnets (760) positioned within housing assembly (732, 734). By way of example only, housing assembly (732, 734) may be formed of a non-ferrous material (e.g., titanium, plastic, etc.). Magnets (760) may be similar in construction and function to any of the aforementioned magnets (160, 260, 360, 460, 660). Each bead (730) is rotatably mounted to a pair of linkage assemblies (740), such that linkage assemblies (740) join beads (730) together.

Each linkage assembly (740) includes a pair of rods (742) that are rotatably joined together via a swivel joint (744). In the present example, each linkage assembly (740) is configured such that each swivel joint (744) is positioned at a middle region defined between adjacent beads (730). In some versions, the end of each rod (742) that is opposite to swivel joint (744) is pivotably coupled with the corresponding bead (730). In some other versions, the end of each rod (742) that is opposite to swivel joint (744) is slidably coupled with the corresponding bead (730). In some other versions, the end of each rod (742) that is opposite to swivel joint (744) is fixedly coupled with the corresponding bead (730).

FIG. 19A shows sphincter augmentation device (720) in a default state, not being acted on by an MRI magnetic field (M). While sphincter augmentation device (720) is not shown as forming a loop shape in FIG. 19A, it should be understood that sphincter augmentation device (720) may be formed in a loop shape about a malfunctioning LES (6). To achieve such a loop shape, sphincter augmentation device (720) may include a clasp feature that secures otherwise-free ends of sphincter augmentation device (720) together. FIG. 19A includes a representation of an opening (770) that would be formed by the loop shape. In the state shown in FIG. 19A, magnets (760) are magnetically attracted to each other due to the orientation of opposing poles (N, S) of magnets (760).

FIG. 19B shows the same components shown in FIG. 19A, but with the MRI machine (MRI) in an activated state such that the MRI machine is generating an MRI magnetic field (M). In response to this MRI magnetic field (M), magnets (760) are magnetically urged to align with the MRI magnetic field (M). Sphincter augmentation device (720) accommodates such alignment by allowing linkage assemblies (740) to pivot at swivel joints (744) and/or at the interfaces between rods (742) and beads (730). Sphincter augmentation device (720) may thus assume an irregular shape in response to the MRI magnetic field (M). The pivotal motion permitted at swivel joints (744) and/or at the interfaces between rods (742) and beads (730) prevents linkage assemblies (740) from being damaged as sphincter augmentation device (720) achieves an irregular shape in response to the MRI magnetic field (M).

With sphincter augmentation device (720) accommodating alignment by allowing linkage assemblies (740) to pivot at swivel joints (744) and/or at the interfaces between rods (742) and beads (730) in response to the presence of an MRI magnetic field (M), sphincter augmentation device (720) may tolerate the presence of the MRI magnetic field (M) without causing any damage to sphincter augmentation device (720). Once the MRI magnetic field (M) is removed (e.g., when the MRI machine (MRI) is deactivated), sphincter augmentation device (720) may readily return to the state shown in FIG. 19A. For instance, once the MRI magnetic field (M) is removed, linkage assemblies (740) may pivot at swivel joints (744) and/or at the interfaces between rods (742) and beads (730) to allow beads (730) to achieve a relative positioning where opposing poles (N, S) of magnets (760) face each other.

### H. Sphincter Augmentation Device with Pivotable and Translatable Linking Structure

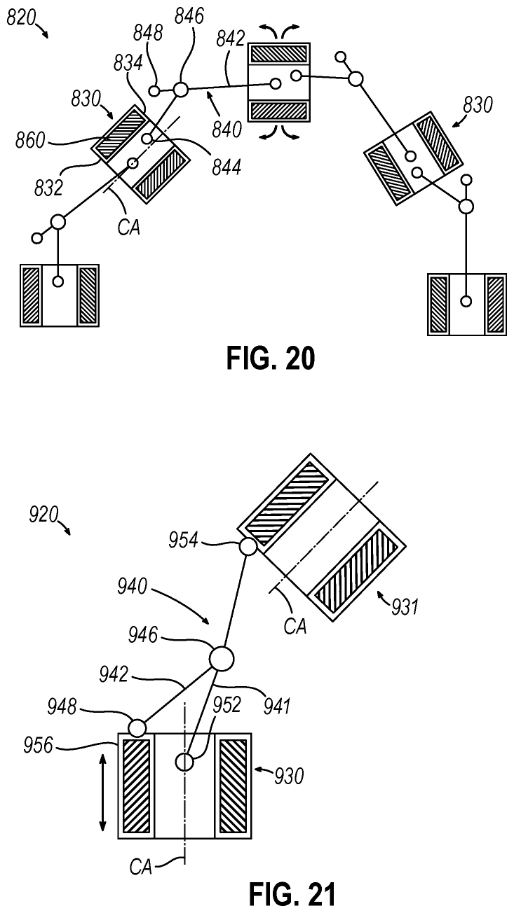

FIG. 20 shows a portion of another exemplary sphincter augmentation device (820) that may be used as an implant around a malfunctioning LES (6), where sphincter augmentation device (820) will not be damaged by an MRI magnetic field (M). Sphincter augmentation device (820) is similar in structure and function to sphincter augmentation device (720) described above except as otherwise described below.

Similar to sphincter augmentation device (720) described above, sphincter augmentation device (820) of this example comprises a plurality of beads (830) that are joined together by respective linkage assemblies (840) to form an annular loop that is configured to be installed around a malfunctioning LES (6). Each bead (830) of sphincter augmentation device (820) includes a housing assembly (832, 834), with one or more magnets (860) positioned within housing assembly (832, 834). By way of example only, housing assembly (832, 834) may be formed of a non-ferrous material (e.g., titanium, plastic, etc.). Magnets (860) may be similar in construction and function to any of the aforementioned magnets (160, 260, 360, 460, 660, 760).

Each linkage assembly (840) includes a pair of rods (841, 842) that are joined together via a joint (846). Each rod (841) includes a first end that is pivotably joined to a housing assembly (832, 834) and a second end that terminates at joint (846). Each rod (842) includes a first end that is pivotably joined to another housing assembly (832, 834) and a second end that terminates in a stop member (848). Rod (842) is slidably received in joint (846), such that joint (846) is slidable along rod (842). In some versions, rod (842) is also pivotable relative to rod (841) at joint (846). Stop member (848) is configured to prevent the second end of rod (842) from passing through joint (846).

While not shown, sphincter augmentation device (820) may also include a clasp feature that secures otherwise-free ends of sphincter augmentation device (820) together, thereby enabling sphincter augmentation device (820) to form a loop around an LES (6). In such a loop formation, magnets (860) may be magnetically attracted to each other due to the orientation of opposing poles (N, S) of magnets (860), such that sphincter augmentation device (820) magnetically biases the LES (60) to achieve a closed state as described above.

In the event that sphincter augmentation device (820) encounters an MRI magnetic field (M) due to the presence of an activated MRI machine (MRI), magnets (860) may be magnetically urged to align with the MRI magnetic field (M). Sphincter augmentation device (820) accommodates such alignment by allowing rods (841) to pivot at beads (830), by allowing rods (842) to pivot at beads (830), and/or by allowing rod (842) to pivot and/or slide relative to rod (841) at joint (846). Sphincter augmentation device (820) may thus assume an irregular shape in response to the MRI magnetic field (M). The slidable and/or pivotal motion permitted joint (846) and/or the pivotal motion permitted at the interfaces between rods (841, 842) and beads (830) prevents linkage assemblies (840) from being damaged as sphincter augmentation device (820) achieves an irregular shape in response to the MRI magnetic field (M).

With sphincter augmentation device (820) accommodating alignment by allowing rods (841) to pivot at beads (830), by allowing rods (842) to pivot at beads (830), and/or by allowing rod (842) to pivot and/or slide relative to rod (841) at joint (846), in response to the presence of an MRI magnetic field (M), sphincter augmentation device (820) may tolerate the presence of the MRI magnetic field (M) without causing any damage to sphincter augmentation device (820). Once the MRI magnetic field (M) is removed (e.g., when the MRI machine (MRI) is deactivated), sphincter augmentation device (820) may readily return to a default, loop-shaped state as described herein. For instance, once the MRI magnetic field (M) is removed, rods (841) may pivot at beads (830), rods (842) may pivot at beads (830), and/or by rod (842) may pivot and/or slide relative to rod (841) at joint (846) to allow beads (830) to achieve a relative positioning where opposing poles (N, S) of magnets (860) face each other.

### I. Sphincter Augmentation Device with Pivotable and Translatable Linking Structure with Beads Connected with Multiple Wires

FIG. 21 shows a portion of another exemplary sphincter augmentation device (920) that may be used as an implant around a malfunctioning LES (6), where sphincter augmentation device (920) will not be damaged by an MRI magnetic field (M). Sphincter augmentation device (920) is similar in structure and function to sphincter augmentation device (820) described above except as otherwise described below.

Sphincter augmentation device (920) of this example comprises a plurality of beads (930) that are joined together by respective linkage assemblies (940) to form an annular loop that is configured to be installed around a malfunctioning LES (6). Each bead (930) of sphincter augmentation device (920) includes a housing assembly (932, 934), with one or more magnets (960) positioned within housing assembly (932, 934). By way of example only, housing assembly (932, 934) may be formed of a non-ferrous material (e.g., titanium, plastic, etc.). Magnets (960) may be similar in construction and function to any of the aforementioned magnets (160, 260, 360, 460, 660, 760, 860).

Each linkage assembly (940) includes a pair of rods (941, 942) that are joined together via a joint (946). Each rod (941) includes a first end that is pivotably joined to one housing assembly (932, 934) and a second end that is pivotably joined to another housing assembly (932). Each rod (942) includes a first end that is pivotably and slidably joined to a housing assembly (932, 934) via a slide coupling (948); and a second end that terminates at joint (946). Rod (941) is slidably received in joint (946), such that joint (946) is slidable along rod (941). Rod (942) is also pivotable relative to rod (941) at joint (946). In some versions, one or both of rods (941, 942) is/are rigid or semi-rigid.

While not shown, sphincter augmentation device (920) may also include a clasp feature that secures otherwise-free ends of sphincter augmentation device (920) together, thereby enabling sphincter augmentation device (920) to form a loop around an LES (6). In such a loop formation, magnets (960) may be magnetically attracted to each other due to the orientation of opposing poles (N, S) of magnets (960), such that sphincter augmentation device (920) magnetically biases the LES (60) to achieve a closed state as described above.

In the event that sphincter augmentation device (920) encounters an MRI magnetic field (M) due to the presence of an activated MRI machine (MRI), magnets (960) may be magnetically urged to align with the MRI magnetic field (M). Sphincter augmentation device (920) accommodates such alignment by allowing rods (941) to pivot at beads (930), by allowing rods (942) to pivot and slide relative to beads (930) at slide couplings (948), and/or by allowing rods (942) to pivot and/or slide relative to rods (941) at joints (946). Sphincter augmentation device (920) may thus assume an irregular shape in response to the MRI magnetic field (M). The slidable and/or pivotal motion permitted joint (946), the pivotal motion permitted at the interfaces between rods (941) and beads (930), and/or the pivotal and sliding motion permitted at slide coupling (948) prevents linkage assemblies (940) from being damaged as sphincter augmentation device (920) achieves an irregular shape in response to the MRI magnetic field (M).

With sphincter augmentation device (920) accommodating alignment by allowing rods (941) to pivot at beads (930), by allowing rods (942) to pivot and slide relative to beads (930) at slide couplings (948), and/or by allowing rods (942) to pivot and/or slide relative to rods (941) at joints (946), in response to the presence of an MRI magnetic field (M), sphincter augmentation device (920) may tolerate the presence of the MRI magnetic field (M) without causing any damage to sphincter augmentation device (920). Once the MRI magnetic field (M) is removed (e.g., when the MRI machine (MRI) is deactivated), sphincter augmentation device (820) may readily return to a default, loop-shaped state as described herein. For instance, once the MRI magnetic field (M) is removed, rods (941) may pivot at beads (930), rods (942) may pivot and slide relative to beads (930) at slide couplings (948), and/or rods (942) may pivot and/or slide relative to rods (941) at joints (946), to allow beads (930) to achieve a relative positioning where opposing poles (N, S) of magnets (960) face each other.

### III. FLEXIBLE ENDOSCOPE AND METHOD OF MEASURING LES AND DEPLOYING SPHINCTER AUGMENTATION DEVICE

The size of an LES (6) may vary from patient to patient, such that it may be desirable to determine the size of a patient's LES (6) to determine an appropriate size of a sphincter augmentation device (20) for that patient. Some LES (6) sizing techniques may include the use of a laparoscope (e.g., inserted through the abdomen of the patient) to visually observe the diameter of the LES (6). Some methods of installing a sphincter augmentation device (20) may include inserting sphincter augmentation device (20) via a trocar, incision, or other access port formed through the abdomen of the patient. As will be described in greater detail below, some variations of an endoscope may be used to size the patient's LES (6), and/or to install sphincter augmentation device (20), from within the esophagus (2) of the patient.

### A. Flexible Endoscope with Pressure Sensitive Bladders

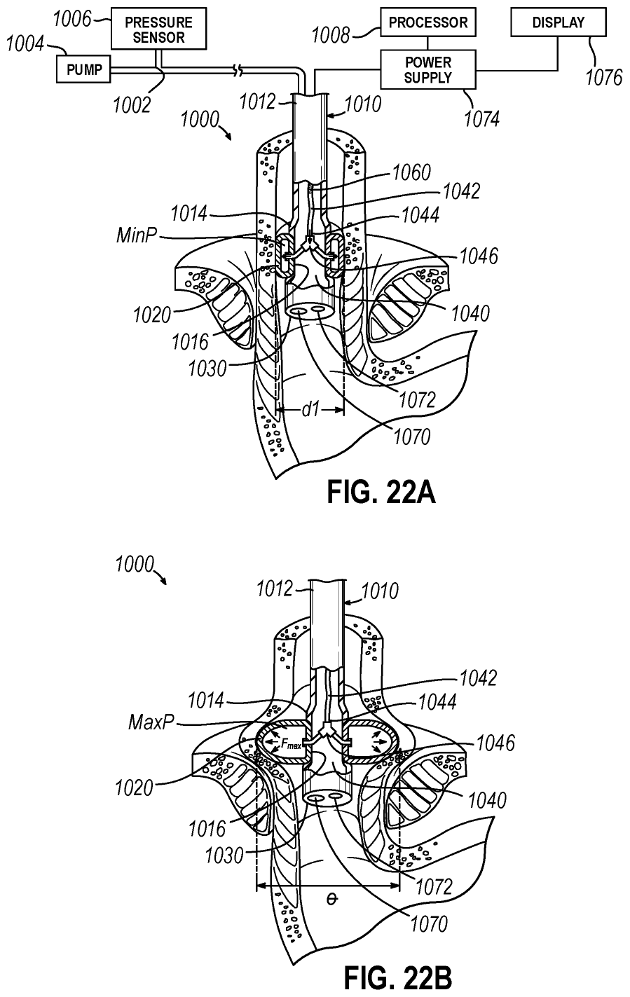

FIG. 22A shows a flexible endoscope (1000) comprising a flexible shaft (1010) extending from a proximal portion (1012) to a distal portion (1014) and at least one inflatable bladder (1020) positioned on an external surface (1016) of the distal portion (1014). Flexible shaft (1010) and inflatable bladder (1020) are shown after being inserted through the mouth of a patient through the esophagus (2) of the patient, such that distal portion (1014) is positioned adjacent to LES (6) with bladder (1020) in a non-expanded state.

Distal portion (1014) of flexible shaft (1010) is positioned between a distal end (1030) of flexible shaft (1010) and the proximal portion (1012). Distal portion (1014) may be sized slightly larger relative to the proximal portion (1012), though this is not necessary in all versions. Distal portion (1014) of flexible shaft (1010) is sized to fit within the esophagus (2) of the patient and inflatable bladder (1020) may be recessed within the outside profile of distal portion (1014) on external surface (1016), so that a deflated diameter (d1) of the inflatable bladder (1020) is reduced when inflatable bladder (1020) is in deflated state for ease of passage through the mouth and esophagus (2) of the patient. In the present example, only one annular or toroidal shaped bladder (1020) is provided at distal portion (1014), with bladder (1020) fully encircling distal portion (1014). In some other versions, distal portion (1014) may include two or more bladders having any suitable configuration and arrangement. Flexible shaft (1010) further includes a shaft lumen (1040) and a bladder lumen (1042) positioned within the shaft lumen (1040). Bladder lumen (1042) is in fluid communication with inflatable bladder (1020). Bladder lumen (1042) extends distally within shaft lumen (1040) from a proximal end (not shown) to a distal end (1044). Distal manifold (1046) is in fluid communication with distal end (1044) of bladder lumen (1042). Distal manifold (1046) evenly distributes working fluid (1060) to inflatable bladder (1020).

The proximal end of bladder lumen (1042) is configured to fixedly couple to a proximal manifold (1002); and distal end (1044) of bladder lumen (1042) is configured to fixedly couple to a distal manifold (1046). Proximal manifold (1002) is in fluid communication with a pump (1004) and a pressure sensor (1006). Pump (1004) is configured to producing a range of pressures with a working fluid (1060), such as air or saline, to transition inflatable bladder (1020) from a deflated state to an expanded state. Pressure sensor (1006) is configured to indicate to a user the pressure administered to inflatable bladder (1020). Pressure sensor (1006) may include a gauge or electronic sensor configured to indicate to a user the pressure within bladder lumen (1042).

In versions where pressure sensor (1006) includes an electronic sensor, the electronic sensor may be in electrical communication with a processor (1008) that interpolates the signal from the electronic sensor to determine the size of the internal diameter of the LES (6). Processor (1008) is in electrical communication with an indicator such as a digital, analog, or virtual display so that a user may reference data collected by the electronic pressure sensor (1006). In the present illustration, inflatable bladder (1020) is inflated to a minimum pressure (MIN P) via the pump (1004), proximal manifold (1002), bladder lumen (1042), and distal manifold (1046). Inflatable bladder (1020) is partially inflated to a minimal pressure (MIN P) that corresponds with a minimum force to define the small diameter without any excessive pressure being applied to inflatable bladder (1020); such that bladder (1020) does not dilate the LES (6). Pressure of partial inflation may be indicated by indicator in the already-interpolated state to show the minimal force, or directly as minimum pressure.

In the present example, flexible shaft (1010) additionally includes a light (1072) in electrical communication with a power source (1074), one or more cameras (1070) in electrical communication with power source (1074), and a display (1076). Light (1072) is configured to illuminate so that camera (1070) may capture an image viewed from the distal end (1030) of the flexible shaft (1010) as shown; or viewing proximally from the distal portion so that inflatable bladder (1020) and the position of the inflatable bladder (1020) may be viewed from display (1076) so that a user may orient the bladder in a position to analyze the tissue of the LES (6).

FIG. 22B shows flexible endoscope (1000) with flexible shaft (1010) having inflatable bladder (1020) positioned adjacent to the LES (6), with the inflatable bladder (1020) in a fully inflated state. Pump (1004) produces a maximum pressure (Max P) that is delivered via the proximal manifold (1002), lumen (1040), and distal manifold (1046) to transition inflatable bladder (1020) to a fully inflated state. In the fully inflated state, a sensed pressure may be recorded by pressure sensor (1006). The sensed pressure may serve as a proxy for a restriction force at the LES (6), which may in turn be used to determine the magnet force needed from a successful sphincter augmentation device (20). In versions where pressure sensor (1006) includes an electronic sensor as described above, processor (1008) may indicate the sensed pressure and/or the corresponding magnet force to the end user, such that the end user may use this information to appropriately select and/or configure a sphincter augmentation device (20) for the patient at hand.

### B. Flexible Endoscope for Deploying Sphincter Augmentation Device from Intra-Esophageal Approach

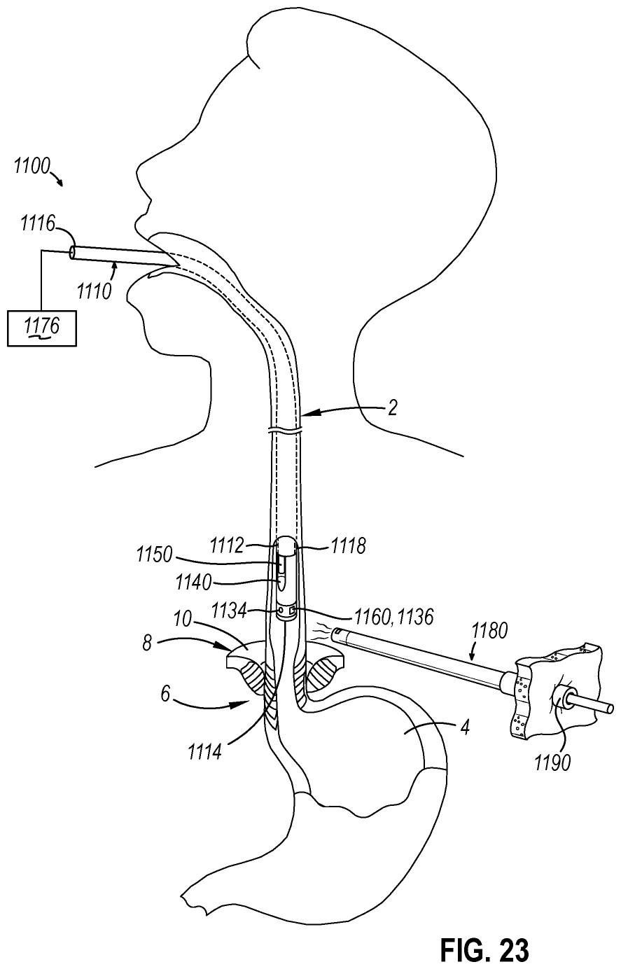

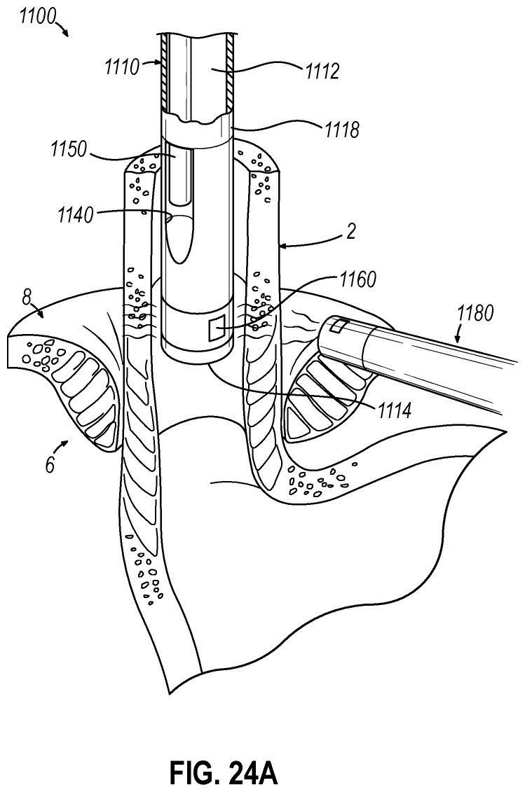

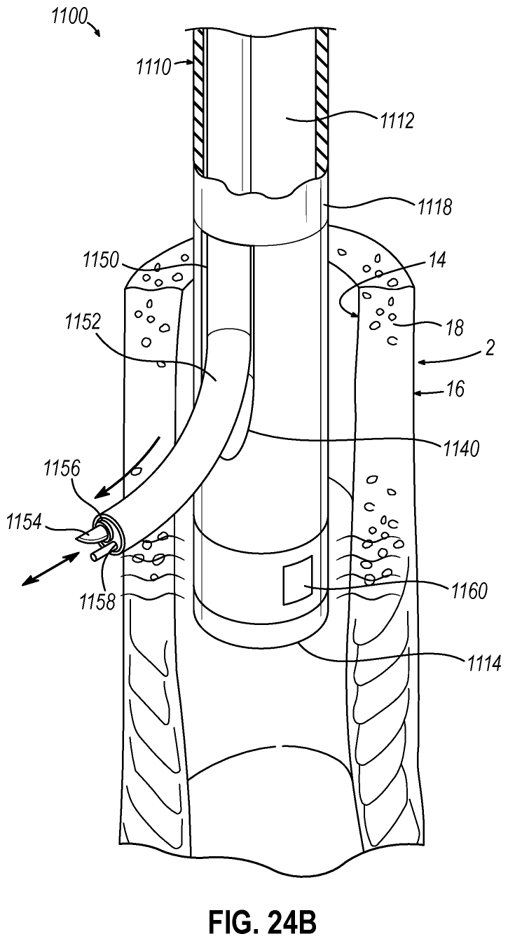

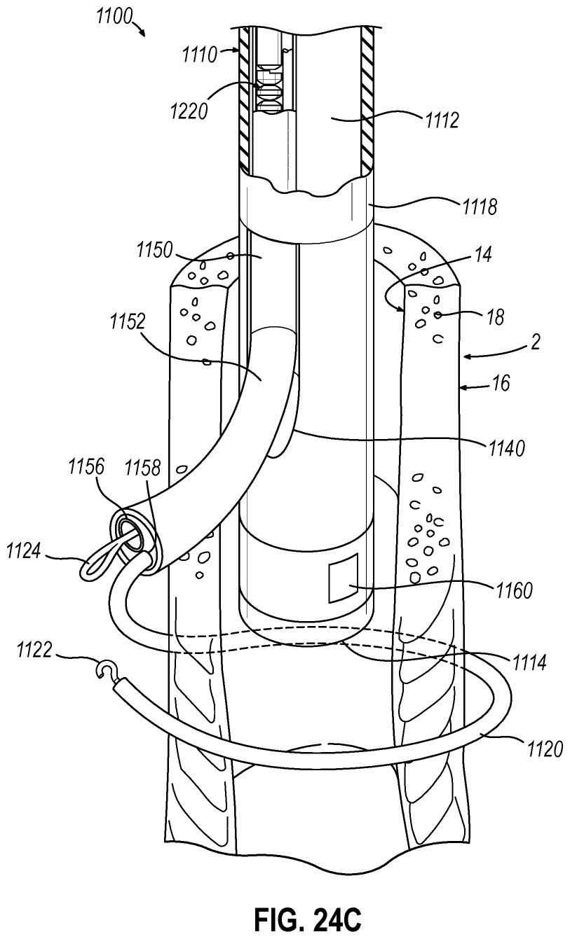

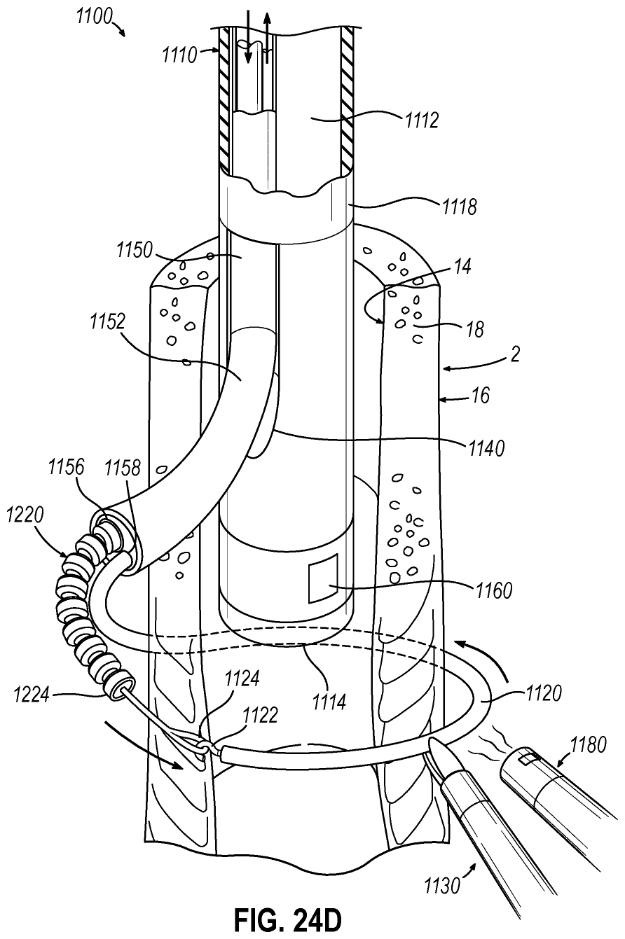

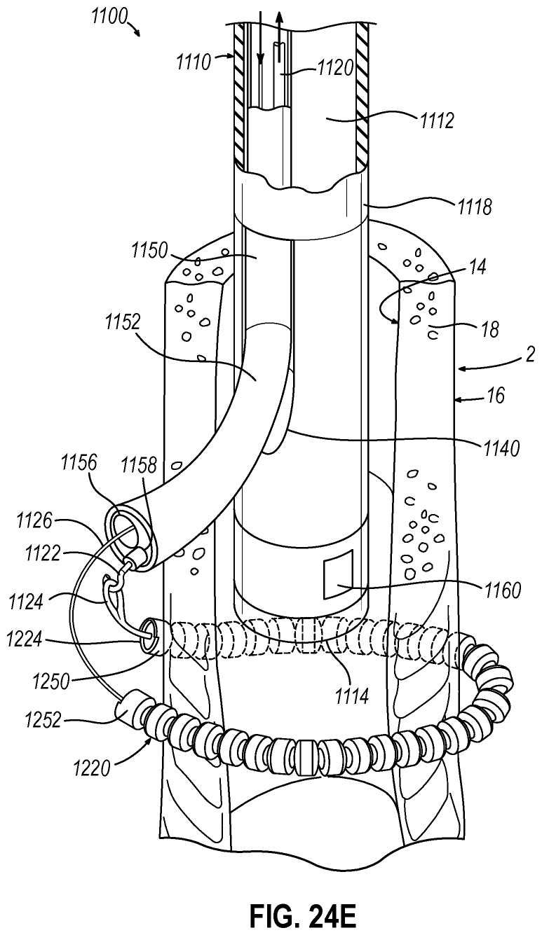

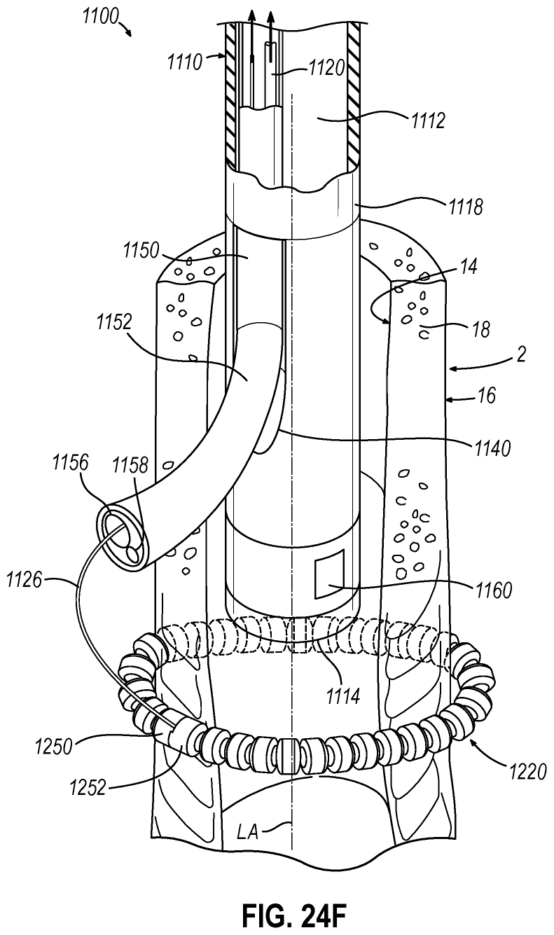

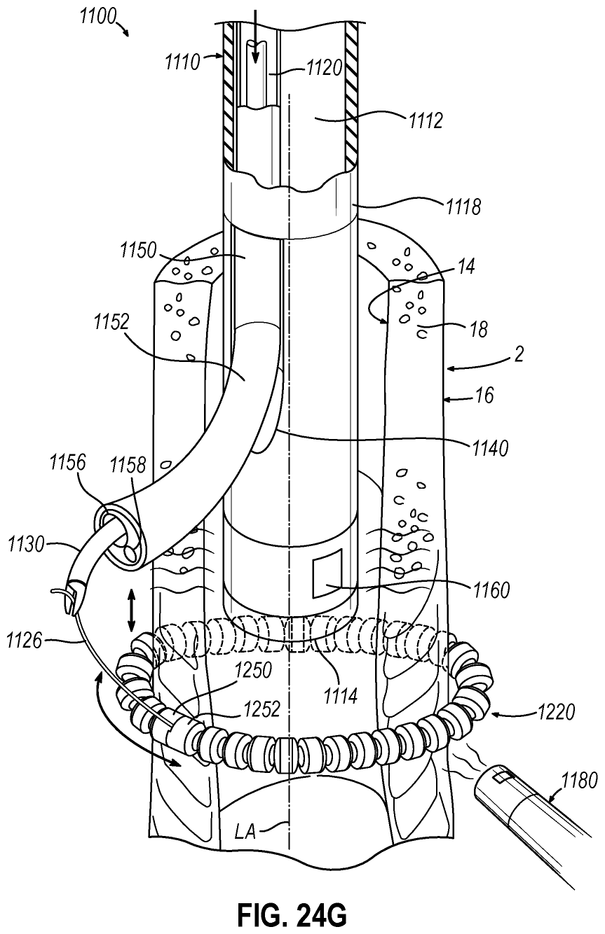

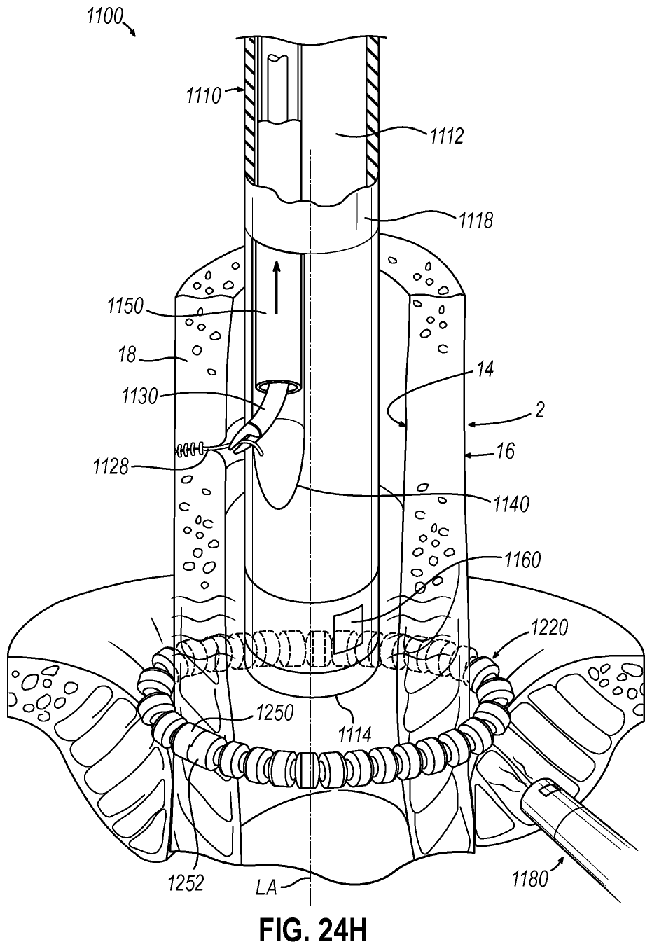

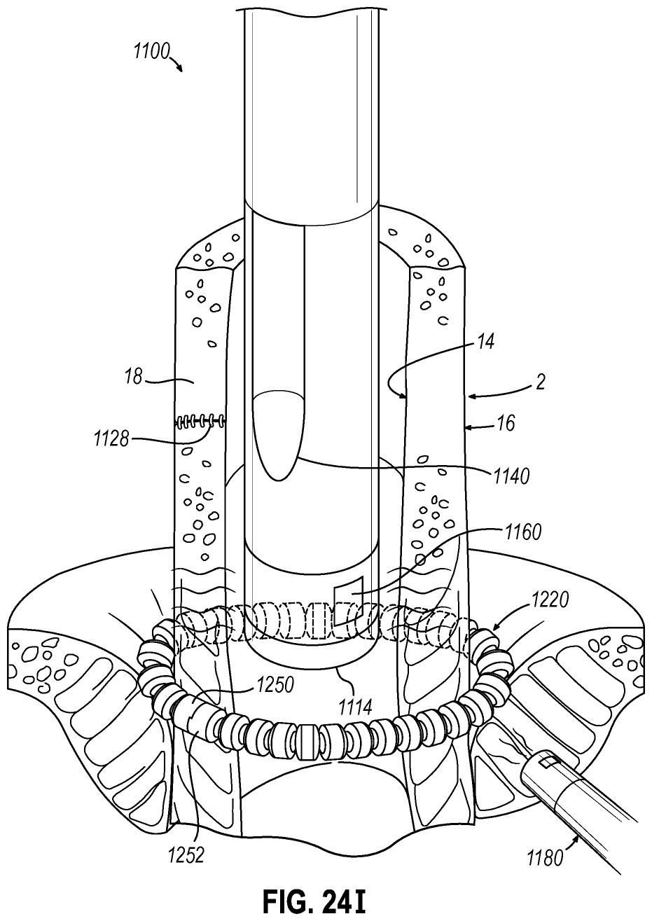

In some instances, it may be desirable to install a sphincter augmentation device (1220) around an exterior of a working channel of the body by accessing the exterior of the working channel of the body from the interior of the working channel of the body, such as by going out from the interior of the esophagus (2) to access the outside of the LES (6). FIGS. 23 and 24A-24I show an example of a flexible endoscope (1100) being used to deploy a sphincter augmentation device (1220) through a sidewall of the esophagus (2) of the patient. In some versions, endoscope (1100) may include all of the structures and functionalities of endoscope (1000), described above, in addition to the structures and functionalities described below. Endoscope (1100) of this example includes a flexible shaft (1110) extending distally from a proximal end (1116) to a distal end (1114), an infrared multispectral imaging camera (1160) positioned on a distal portion (1118), a transverse port (1140) proximal of distal end (1114), and a sleeve (1150) removably positioned within flexible shaft (1110). FIG. 23 also shows a laparoscope (1180) being inserted through a small incision in the body through a trocar (1190).

Flexible shaft (1110) includes a shaft lumen (1112) that is configured to receive a removable sleeve (1150) within shaft lumen (1112). Flexible shaft (1110) may be constructed of a medically safe material such as titanium, stainless steel, rubber, or plastic and may include features enabling flexible shaft (1110) to bend in order to navigate through the esophagus (2). Flexible shaft (1110) includes a working length that measures from the proximal end (1116) to the distal end (1114) so that the flexible shaft (1110) may be inserted through a mouth of a patient and reach the LES (6) via the esophagus (2). Flexible shaft (1110) of the present example further includes a light (1134) (e.g., an LED, etc.) that is capable of producing light to guide distal end (1114) of the flexible shaft (1110) through the esophagus (2). Flexible shaft (1110) may also include a conventional camera (1136) located on a distal portion (1118) configured to capture red, green, blue (“RBG”) images. Conventional camera (1136) is in electrical communication with a display (1076) that is configured to display an image of the esophagus (2) to facilitate guidance of the flexible shaft (1110) through the esophagus (2).

Infrared multi spectral imaging camera (1160) is also in electrical communication with display (1176). Infrared multispectral imaging camera (1160) is configured to use hyper-spectral imaging to capture more than one image of different wavebands of non-visible near-infrared (“NIR”) channels and are overlaid with an RBG image to prove a user with an “augmented” view of the tissues or blood vessels within the esophagus (2) or adjacent physiological structures of the body. The use of infrared multi spectral imaging camera (1160) may be supplemented by a patient being injected or ingesting a dye to further view physiological structures of the body.

Distal portion (1118) of flexible shaft (1110) further includes transverse port (1140) disposed in a sidewall of the flexible shaft (1110). Transverse port (1140) includes an arcuate distal shape and is configured to receive and support sleeve (1150) therethrough. Sleeve (1150) is advanceable relative to flexible shaft (1110) such that a distally located bent portion (1152) may protrude out from flexible shaft (1110). The distal end of bent portion (1152) includes a sharp obturator (1154). Bent portion (1152) is sized to fit within the shaft lumen (1112). In some versions, sleeve (1150) is steerable such that bent portion (1152) maintains a straight configuration when bent portion (1152) is disposed within shaft lumen (1112) (FIG. 24A); yet the operator may activate an actuator (e.g., pull-wire, etc.) to drive bent portion (1152) to the bent state after bent portion (1152) exits shaft lumen (1112) via transverse port (1140) (FIG. 24B). In some other versions, sleeve (1150) is configured such that bent portion (1152) is resiliently biased to bend such that bent portion (1152) maintains a straight configuration under stress when bent portion (1152) is disposed within shaft lumen (1112) (FIG. 24A); yet the resilience of bent portion (1152) drives bent portion (1152) to the bent state after bent portion (1152) exits shaft lumen (1112) via transverse port (1140) (FIG. 24B). Alternatively, bent portion (1152) may have any other suitable configuration or form of operation.

Sleeve (1150) further includes a large lumen (1156) and a small lumen (1158). Large lumen (1156) is separated from small lumen (1158) by an arcuate inner wall. Both small and large lumens (1156, 1158) terminate at the sharp obturator (1154). Sharp obturator (1154) is configured to pierce through a sidewall of esophagus (2) as described below.

FIG. 24A shows flexible shaft (1110) being inserted distally through the esophagus (2) of the patient to a position proximate to the LES (6). Once proximate to the LES (6) through the aid of laparoscope (1180), infrared multispectral imaging camera (1160) is used to analyze the sidewall of the esophagus (2) to determine a suitable place to make an incision in the sidewall of the esophagus (2). In some procedures, flexible endoscope (1000) may be inserted in a previous step to appropriately size a sphincter augmentation device (1220) for the patient at hand and determine the correct magnetic attraction to install around the LES (6) before inserting the flexible shaft (1110) of flexible endoscope (1100) into the esophagus (2). Sphincter augmentation device (1220) may be configured and operable like any of the various sphincter augmentation devices described herein.

Once the appropriate location for an incision in the sidewall of the esophagus (2) has been determined using laparoscope (1180) and/or infrared multispectral imaging camera (1160), sleeve (1150) is advanced distally relative to shaft (1110), such that bent portion (1152) protrudes from transverse port (1140) as shown in FIG. 24B. As sleeve (1150) is advanced distally through transverse port (1140), sharp obturator (1154) pierces through esophagus (2) to provide access the exterior of esophagus (2), making an incision in the esophagus (2) that extends from the interior sidewall (14) of esophagus (2) to the exterior sidewall (16) of esophagus (2). During the creation of the incision, transverse port (1140) supports and guides bent portion (1152) so that radial force may be exhibited upon the inner sidewall (14) of the esophagus (2).

After obturator (1154) has been used to laterally pierce through the esophagus (2), a guidewire (1120) may be deployed distally through distal end of small lumen (1158) of sleeve (1150), as shown in FIG. 24C. Guidewire (1120) of this example includes a distal hook (1122). As also shown in FIG. 24C, a ribbon loop (1124) is attached to a first end (1224) of a sphincter augmentation device (1220), which is being deployed through a distal end of large lumen (1156). Ribbon loop (1124) may be constructed of an absorbable or non-absorbable material. Guidewire (1120) is driven to encircle the esophagus (2) and thereby position distal hook (1122) near ribbon loop (1124). By way of example only, guidewire (1120) may include one or more pull-wires and/or other features that enable guidewire (1120) to be driven to encircle the esophagus (2) and thereby position distal hook (1122) near ribbon loop (1124). In addition, or in the alternative, additional instrumentation (e.g., graspers, etc.) may be used to assist in positioning distal hook (1122) near ribbon loop (1124).

Distal hook (1122) is then engaged with ribbon loop (1124) as shown in FIG. 24D. In some cases, such engagement may be achieved utilizing one or more pull-wires and/or other features that enable guidewire (1120) to be driven to encircle the esophagus (2). In some other cases, additional instrumentation such as graspers (1130) shown in FIG. 24D are used to assist in achieving engagement between distal hook (1122) and ribbon loop (1124). In either scenario, laparoscope (1180) may provide visualization to facilitate such engagement. Once hook (1122) of guidewire (1120) is coupled with ribbon loop (1124) at a first end (1224) of sphincter augmentation device (1220), guidewire (1120) is retracted proximally through small lumen (1158). Such retraction of guidewire (1120) pulls sphincter augmentation device (1220) distally through large lumen (1156). Guidewire (1120) follows a path around exterior sidewall (16) of esophagus (2) as guidewire (1120) is retracted proximally, such that guidewire (1120) pulls sphincter augmentation device (1220) around exterior sidewall (16) of esophagus (2) as guidewire (1120) is retracted proximally.

FIG. 24E shows guidewire (1120) being further retracted through small lumen (1158) until sphincter augmentation device (1220) is positioned around the exterior sidewall (16) of the esophagus (2). Sphincter augmentation device (1220) is fully deployed from the large lumen (1156) at this stage. A second end of sphincter augmentation device (1220) is affixed to a suture (1126), which extends through a length of the large lumen (1156). Suture (1126) may be constructed of an absorbable or non-absorbable material. With sphincter augmentation device (1220) being fully deployed from large lumen (1156) at this stage, suture (1126) is also exposed from large lumen (1156).

After sphincter augmentation device (1220) is fully deployed from large lumen (1156) and encircles the esophagus (2), the free ends of sphincter augmentation device (1220) are joined together. This may be achieved by simultaneously retracting guidewire (1120) and suture (1126) proximally through small lumen (1158) and large lumen (1156), respectively, as shown in FIG. 24F. A first joining bead (1250) is positioned on first end (1224) of sphincter augmentation device (1220) and a second joining bead (1252) positioned on a second end (1226) of sphincter augmentation device (1220). First joining bead (1250) is magnetically attracted to second joining bead (1252) and both join together in a clasping arrangement such that sphincter augmentation device (1220) forms an annular loop around the exterior sidewall (16) of esophagus (2). It should be noted that joining beads (1250, 1252) may be joined together without any further interaction and may be joined together for the entire time period that the sphincter augmentation device (1220) remains in the body. Any suitable kinds of coupling features may be used to join free ends of sphincter augmentation device (1220) together to form a loop.

FIG. 24G shows a flexible grasper (1130) being extended distally through sleeve (1150) and extending out the distal end of the large lumen (1156). Grasper (1130) engages the suture (1126) to manipulate the sphincter augmentation device (1220) by manipulating the grasper (1130) so that suture (1126) is pulled radially away from the exterior sidewall (16) of the esophagus (2) to enable the sphincter augmentation device (1220) to be moved proximally or distally along a longitudinal axis (LA) of esophagus (2) to a proper location that is best suited to install sphincter augmentation device (1220) to aid a malfunctioning LES (6). Infrared multispectral imaging camera (1160) is used in real time to determine if location is best suited for installation of sphincter augmentation device (1220). While grasper (1130) engages suture (1126) to position sphincter augmentation device (1220) at the appropriate location along the esophagus (2) in the present example, in other variations grasper (1130) may engage beads, links, or other features of sphincter augmentation device (1220) to position sphincter augmentation device (1220) at the appropriate location along the esophagus (2).

Once sphincter augmentation device (1220) has been moved to the appropriate location along the esophagus (2), grasper (1130) may release suture (1126) (or some other portion of sphincter augmentation device (1220)) and may then be retracted proximally along large lumen (1156) as shown in FIG. 24H. FIG. 24H also shows sleeve (1150) having been retracted proximally from sidewall (18) of the esophagus (2) and has through shaft lumen (1112). Infrared multispectral imaging camera (1160) is used to further assess the location of the sphincter augmentation device (1220) to confirm that no further repositioning is warranted. Additionally, laparoscope (1180) is used to further confirm whether the sphincter augmentation device (1120) is positioned to best aid the LES (6). After the assessment of the location of the sphincter augmentation device (1120) is complete, the incision in the esophagus (2) is closed by using grasper (1130) and a suture thread (1128). In the present example, this suturing is performed from within the esophagus (2) by grasper (1130). In some other variations, this suturing is performed via a grasper and suture introduced to the exterior of esophagus (2) via a trocar or other access port disposed in the patient's abdomen. While a grasper (1130) and suture are used in the present example, other instrumentation and techniques may be used to close the incision in the esophagus (2).

FIG. 24I shows the sphincter augmentation device (1220) installed in a proper location to aid the LES (6) with the flexible shaft (1110) removed and the incision closed with suture thread (1128). Laparoscope (1180) is again used to double check the location of the sphincter augmentation device (1220) along the esophagus (2). Laparoscope (1180) may also be used to inspect the now-closed incision in esophagus (2). Once this is complete, laparoscope (1180) and trocar (1190) may be removed, and the incision in the abdomen through which trocar (1190) was disposed is closed.

### IV. EXAMPLES OF COMBINATIONS

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A sphincter augmentation device comprising: (a) a plurality of bodies, wherein each of the bodies includes: (i) a housing including a central axis therethrough, and (ii) a plurality of magnets positioned within the housing, wherein the magnets are configured to magnetically bias the bodies toward one another, wherein the magnets are configured to move within the housing; and (b) a linking structure configured to link the plurality of bodies together to form the bodies in an annular array; wherein the annular array is sized to be positioned around a human lower esophageal sphincter so that the bodies and the linking structure bear inwardly against the lower esophageal sphincter; wherein the annular array is configured to transition between a radially expanded state and a radially contracted state to constrict the lower esophageal sphincter; wherein the magnets of each body are configured to move relative to the housing of each body between a first position and a second position, wherein in the first position the magnets are magnetically aligned with each other, wherein in the second position the magnets are aligned with an externally applied magnetic field.

### Example 2

The sphincter augmentation device of Example 1, wherein the magnets of each body are positioned within the housing in an annular array about the central axis.

### Example 3

The sphincter augmentation device of Example 2, wherein each magnet of the plurality of magnets has a spherical shape.

### Example 4

The sphincter augmentation device of any of Examples 1 through 3, wherein the magnets of each body are positioned within the housing in a pair of coaxial annular arrays about the central axis.

### Example 5

The sphincter augmentation device of any of Examples 1 through 4, wherein the housing includes a dampening feature configured to reduce movement of the magnets relative to the housing when transitioning from the first position to the second position.

### Example 6

The sphincter augmentation device of Example 5, wherein the dampening feature comprises polypropylene.

### Example 7

The sphincter augmentation device of any of Examples 1 through 6, wherein the housing includes two end caps and a middle section, wherein the end caps are constructed of a different material than the middle section.

### Example 8

The sphincter augmentation device of Example 7, wherein the middle section is constructed of a ferromagnetic material and the two end caps are constructed of a non-ferromagnetic material.

### Example 9

The sphincter augmentation device of any of Examples 1 through 8, wherein the linking structure includes a flexible band, wherein the flexible band is elastic and is configured to transition between a first state when not acted upon by an external magnetic field and a second state when acted upon by the external magnetic field, wherein the flexible band in the second state is configured to allow the plurality of bodies to align with the external magnetic field, wherein when no longer acted upon by the external magnetic field the flexible band is configured to return to the first state without damaging or distorting the flexible band.

### Example 10

The sphincter augmentation device of any of Examples 1 through 9, wherein the linking structure includes a flexible band having a first end and a second end, wherein the first and second ends are slidably linked together.

### Example 11

The sphincter augmentation device of Example 10, wherein the first end is located on a first portion of the flexible band and the second end is located on a second portion of the flexible band, wherein the first portion is configured to slide within the second portion of the flexible band.

### Example 12

The sphincter augmentation device of any of Examples 1 through 11, wherein the linking structure includes a plurality of bands, wherein each of the bands includes a first end and a second end, wherein the first ends are configured to slidably link with the second ends.

### Example 13

The sphincter augmentation device of Example 12, wherein the first end is located on a first portion of each band and the second end is located on a second portion of each band, wherein the first portion of each band is configured to slide within the second portion of each band.

### Example 14

The sphincter augmentation device of any of Examples 1 through 13, wherein the central axis of the housing is offset from the linking structure.

### Example 15

The sphincter augmentation device of Example 14, wherein the housing is rotatably coupled to the linking structure with a pin extending transversely though the linking structure and retained by a tab on a side opposite the housing.

### Example 16

A sphincter augmentation device comprising: (a) a first body including a first housing and a first magnet; (b) a second body including a second housing and a second magnet; and (c) a linking structure configured to link the first body to the second body; wherein the sphincter augmentation device is sized to be positioned around a human lower esophageal sphincter so that the first and second bodies bear inwardly against the lower esophageal sphincter; wherein the sphincter augmentation device is configured to transition from an expanded state to a contracted state by magnetic attraction of the first and second magnets to constrict the lower esophageal sphincter; wherein the first and second magnets are configured to transition from a first position to a second position when an external magnetic field acts upon the first and second magnet, such that the first and second magnets are configured to align with the external magnetic field in the second position; wherein the bodies or linking structure are configured to transition from the second position to the first position without being damaged.

### Example 17

The sphincter augmentation device of Example 16, wherein the magnets are configured to move within the housings to align with the external magnetic field.

### Example 18

The sphincter augmentation device of Example 17, wherein the magnets have a spherical shape.

### Example 19

A sphincter augmentation device comprising: (a) a plurality of bodies, wherein each body of the plurality of bodies includes: (i) a housing including a central axis therethrough and a ferromagnetic middle portion such that the middle portion is configured to damp an external magnetic field, and (ii) one or more magnets positioned within the housing, wherein the magnets are configured to magnetically bias the bodies toward one another; and (b) a linking structure configured to link the plurality of bodies together to form the bodies in an annular array; wherein the annular array is sized to be positioned around a human lower esophageal sphincter so that the bodies and the linking structure bear inwardly against the lower esophageal sphincter; wherein the annular array is configured to transition between a radially expanded state and a radially contracted state to constrict the lower esophageal sphincter.

### Example 20

The sphincter augmentation device of Example 19, wherein the housing further includes non-ferromagnetic end portions configured to allow magnetic attraction between adjacent beads, wherein the middle portion includes a ferromagnetic coating configured to damp an external magnetic field.

### Example 21

An apparatus comprising: (a) a first shaft extending distally from a proximal end to a distal end, wherein the first shaft is sized to fit within an esophagus of a patient, wherein the first shaft includes a first shaft lumen extending distally to the distal end; and (b) a second shaft slidably positioned within the first shaft lumen, wherein the second shaft is sized to fit within the shaft lumen and extend through the transverse bore, wherein the second shaft is configured to receive a guide element and a sphincter augmentation device so that the sphincter augmentation device may be deployed through an interior of the esophagus to an exterior of the esophagus.

### Example 22

The apparatus of Example 21, wherein the second shaft includes a second lumen sized to receive the sphincter augmentation device therethrough.

### Example 23

The apparatus of Example 22, wherein the second lumen is further configured to receive a grasper therethrough.

### Example 24

The apparatus of any of Examples 21 through 23, wherein the second shaft includes a third lumen sized to receive the guide element therethrough.

### Example 25

The apparatus of any of Examples 21 through 24, wherein the second shaft includes a sharp obturator configured to penetrate the esophagus to create a pathway for the second shaft to pass through the esophagus to deploy the sphincter augmentation device around the exterior of the esophagus.

### Example 26

The apparatus of Example 25, wherein the first shaft includes a multispectral imaging camera configured to determine a location to penetrate the esophagus with the sharp obturator.

### Example 27

The apparatus of any of Examples 21 through 26, wherein the first shaft includes a light.

### Example 28

The apparatus of any of Examples 21 through 27, wherein the first shaft includes a camera configured to communicate with a monitor to provide an image to aid in navigating the first shaft through the esophagus.

### Example 29

The apparatus of any of Examples 27 through 28, wherein the first shaft includes a working length that extends from the proximal end to the distal end, wherein the proximal end is positionable proximal of a mouth of patient and the distal end is positionable proximate to a lower esophagus sphincter.

### Example 30

The apparatus of any of Examples 21 through 29, wherein the second shaft includes a steerable distal end.

### Example 31

The apparatus of any of Examples 21 through 30, further including the sphincter augmentation device, the sphincter augmentation device having a loop at a first end.

### Example 32

The apparatus of Example 31, further including the guide element, the guide element having a distal hook configured to engage the loop on the first end of the sphincter augmentation device.

### Example 33

The apparatus of Example 32, wherein the guide element is steerable such that a distal portion of the guide element is configured to move by remotely operated controls.

### Example 34

The apparatus of Example 33, wherein the first shaft includes a transverse bore, the second shaft being configured to pass through the transverse bore.

### Example 35

The apparatus of Example 34, the transverse bore including an arcuate distal shape configured to guide and support a distal portion of the second shaft so that the second shaft may exhibit a radial force on an inner sidewall of the esophagus, thereby penetrating the inner sidewall of the esophagus.

### Example 36

An apparatus comprising: (a) a first shaft having a diameter sized to fit within a mouth of a patient and a length sized to extend through the esophagus to a lower esophageal sphincter of the patient; (b) at least one inflatable bladder positioned distally on an external surface of the first shaft, wherein the inflatable bladder is configured to be transitioned from a deflated state to an inflated state, the inflatable bladder being configured to bear outwardly on the lower esophageal sphincter in the inflated state; (c) a lumen operatively connected to the inflatable bladder to provide fluid communication between the lumen and the inflatable bladder; and (d) a pressure sensor operatively connected to the lumen and configured to indicate a pressure that corresponds with a restrictive force when the inflatable bladder is transitioned to the inflated position within the lower esophageal sphincter

### Example 37

The apparatus of Example 36, further including a distal manifold positioned within the first shaft, wherein the distal manifold is configured to provide an equal radial pressure to the at least one inflatable bladder.

### Example 38

A method of deploying a sphincter augmentation device with an apparatus comprising: a first shaft including a shaft lumen having a distal sidewall opening, and a second shaft including a distal bend having a sharp distal end, the method comprising: (a) deploying the first shaft within an esophagus of a patient; (b) deploying the second shaft distally through the shaft lumen and through the distal sidewall opening; (c) piercing an interior sidewall of a patient's esophagus by making an incision with the sharp distal end of the second shaft to gain access to an exterior of the patient's esophagus; and (d) deploying the sphincter augmentation device through a lumen of the second shaft and around the exterior of the patient's esophagus.

### Example 39

The method of Example 38, the apparatus further including a multispectral imaging camera, the method further comprising: (e) analyzing the esophagus and surrounding tissue with the multispectral imaging camera to determine an optimal location to make the incision through the interior sidewall of the esophagus to gain access to the exterior of the esophagus.

### Example 40

The method of Example 39, further comprising: (f) pulling a suture affixed to the sphincter augmentation device through a second shaft lumen to position the sphincter augmentation device in a position around the lower esophageal sphincter.

### V. MISCELLANEOUS

It should also be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/we claim:

1. An apparatus comprising:
(a) a first shaft extending distally from a proximal end to a distal end, wherein the first shaft is sized to fit within an esophagus of a patient, wherein the first shaft includes a first shaft lumen extending distally to the distal end; and
(b) a second shaft slidably positioned within the first shaft lumen, wherein the second shaft is sized to fit within the shaft lumen and extend through the transverse bore, wherein the second shaft is configured to receive a guide element and a sphincter augmentation device so that the sphincter augmentation device may be deployed through an interior of the esophagus to an exterior of the esophagus.

2. The apparatus of claim 1, wherein the second shaft includes a second lumen sized to receive the sphincter augmentation device therethrough.

3. The apparatus of claim 2, wherein the second lumen is further configured to receive a grasper therethrough.

4. The apparatus of claim 1, wherein the second shaft includes a third lumen sized to receive the guide element therethrough.

5. The apparatus of claim 1, wherein the second shaft includes a sharp obturator configured to penetrate the esophagus to create a pathway for the second shaft to pass through the esophagus to deploy the sphincter augmentation device around the exterior of the esophagus.

6. The apparatus of claim 5, wherein the first shaft includes a multispectral imaging camera configured to determine a location to penetrate the esophagus with the sharp obturator.

7. The apparatus of claim 1, wherein the first shaft includes a light.

8. The apparatus of claim 1, wherein the first shaft includes a camera configured to communicate with a monitor to provide an image to aid in navigating the first shaft through the esophagus.

9. The apparatus of claim 7, wherein the first shaft includes a working length that extends from the proximal end to the distal end, wherein the proximal end is positionable proximal of a mouth of patient and the distal end is positionable proximate to a lower esophagus sphincter.

10. The apparatus of claim 1, wherein the second shaft includes a steerable distal end.

11. The apparatus of claim 1, further including the sphincter augmentation device, the sphincter augmentation device having a loop at a first end.

12. The apparatus of claim 11, further including the guide element, the guide element having a distal hook configured to engage the loop on the first end of the sphincter augmentation device.

13. The apparatus of claim 12, wherein the guide element is steerable such that a distal portion of the guide element is configured to move by remotely operated controls.

14. The apparatus of claim 13, wherein the first shaft includes a transverse bore, the second shaft being configured to pass through the transverse bore.

15. The apparatus of claim 14, the transverse bore including an arcuate distal shape configured to guide and support a distal portion of the second shaft so that the second shaft may exhibit a radial force on an inner sidewall of the esophagus, thereby penetrating the inner sidewall of the esophagus.

16. An apparatus comprising:
(a) a first shaft having a diameter sized to fit within a mouth of a patient and a length sized to extend through the esophagus to a lower esophageal sphincter of the patient;
(b) at least one inflatable bladder positioned distally on an external surface of the first shaft, wherein the inflatable bladder is configured to be transitioned from a deflated state to an inflated state, the inflatable bladder being configured to bear outwardly on the lower esophageal sphincter in the inflated state;
(c) a lumen operatively connected to the inflatable bladder to provide fluid communication between the lumen and the inflatable bladder; and
(d) a pressure sensor operatively connected to the lumen and configured to indicate a pressure that corresponds with a restrictive force when the inflatable bladder is transitioned to the inflated position within the lower esophageal sphincter.

17. The apparatus of claim 16, further including a distal manifold positioned within the first shaft, wherein the distal manifold is configured to provide an equal radial pressure to the at least one inflatable bladder.

18. A method of deploying a sphincter augmentation device with an apparatus comprising: a first shaft including a shaft lumen having a distal sidewall opening, and a second shaft including a distal bend having a sharp distal end, the method comprising:
(a) deploying the first shaft within an esophagus of a patient;
(b) deploying the second shaft distally through the shaft lumen and through the distal sidewall opening;
(c) piercing an interior sidewall of a patient's esophagus by making an incision with the sharp distal end of the second shaft to gain access to an exterior of the patient's esophagus; and
(d) deploying the sphincter augmentation device through a lumen of the second shaft and around the exterior of the patient's esophagus.

19. The method of claim 18, the apparatus further including a multispectral imaging camera, the method further comprising:
(e) analyzing the esophagus and surrounding tissue with the multispectral imaging camera to determine an optimal location to make the incision through the interior sidewall of the esophagus to gain access to the exterior of the esophagus.

20. The method of claim 19, further comprising:
(f) pulling a suture affixed to the sphincter augmentation device through a second shaft lumen to position the sphincter augmentation device in a position around the lower esophageal sphincter.
